(19) United States
(12) Patent Application Publication
(10) Pub. No.: US 2010/0317115 A1
(43) Pub. Date: Dec. 16, 2010

(54) METHODS, COMPOSITIONS AND USES FOR ENHANCING CHEMICAL TOLERANCE BY MICROORGANISMS

(76) Inventors: Ryan GILL, Denver, CO (US); Nicholas SANDOVAL, Boulder, CO (US); Tirzah MILLS, Boulder, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/771,845

(22) Filed: Apr. 30, 2010

Related U.S. Application Data
(60) Provisional application No. 61/174,940, filed on May 1, 2009.
(60) Provisional application No. 61/223,322, filed on Jul. 6, 2009.

(51) Int. Cl. C12N 15/70; C12N 15/63; C12N 1/21

(52) U.S. Cl. 435/471; 435/320.1; 435/252.33

(57) ABSTRACT

Embodiments herein concern compositions and methods for enhancing chemical tolerance of biomass conversion by microorganisms. In some embodiments, enhancing tolerance of biomass hydrolysate conversion includes enhancing tolerance to low molecular weight organic compounds.

### CROSS REFERENCE TO RELATED APPLICATIONS

The present application claims the benefit under 35 U.S.C. 119(e) of U.S. Provisional Patent Application Ser. No. 61/174,940 filed on May 1, 2009 and U.S. Provisional Patent Application Ser. No. 61/223,322 filed Jul. 6, 2009, which are incorporated herein by reference in their entirety.

### FEDERALLY FUNDED RESEARCH

Embodiments disclosed herein were supported in part by a grant from the U.S. Department of Energy National Renewable Energy Laboratory and Fellowship number 2007056441 from the National Science Foundation; grant BES0228584 from the National Science Foundation and an Fellowship number 2007056441 from the National Science Foundation. The U.S. government may have certain rights to practice the subject invention.

### FIELD

Embodiments herein report methods, compositions and uses for enhancing tolerance by microorganisms to toxic byproducts from biomass hydrolysis. This application also generally reports methods, compositions and uses of vectors or genetic manipulations to increase the production of industrial fermentation/bioprocesses. In certain embodiments, compositions and methods herein concern biomass conversion into biofuels, production of recombinant proteins for pharmaceutical application or other products generated by a microorganism. Certain embodiments relate to compositions and methods for enhancing tolerance to toxic byproduct molecules such as low molecular weight organic compounds by microorganisms by various methods and compositions. In other embodiments, intracellular levels of certain aldehydes or acetates are modulated to increase production of useful microbial byproducts.

### BACKGROUND

Mass production of useful chemicals can produce problematic byproducts to platform organisms capable of producing these chemicals. Bacteria are capable of producing useful chemicals but often production is hindered by toxic byproducts. Escherichia coli are a well studied microorganism with a completed genome sequence commonly used in the chemical industry. However, approximately 60% of predicted genes in the genome have unknown function.

Cellulosic biomass, for example, cellulose and hemicellulose, include about 75 percent of all plant material. This material can be used as a low-grade fuel that can be burned. Currently it is difficult and costly to turn cellulosic biomass into a biofuels such as a liquid fuel like ethanol. Cellulose and hemicellulose are polymers of sugar, but they are complex compounds not easily broken down into their simpler component sugars. Potential sources of cellulosic biomass include agricultural plant wastes, plant wastes from industrial processes (sawdust, paper pulp), and crops grown specifically for fuel production, such as switchgrass and poplar trees.

Lignocellulosic feedstocks, such as switchgrass, poplar, and corn stover, provide green house gas savings of 65-100% in comparison to petrol. Feedstocks that do not require a substantial change in land-use include crop and municipal wastes, fall grass harvests, and algae. Other potential feedstocks include waste from pulp and paper mills, construction debris, and animal manures. These feedstocks are of extreme interest because they require no additional land-use conversion.

### SUMMARY

Some embodiments herein concern modulating or conferring and/or inducing tolerance of toxic side products of biomass hydrolysis upon a microorganism. Other embodiments herein concern modulating, conferring and/or inducing genes to increase export or induce metabolism of chemical byproducts such as low molecular weight organic compounds (e.g. acetates, aldehydes for example furfurals) in a microorganism. In accordance with these embodiments, microorganisms can increase production of useful chemicals (e.g. by induction of growth, metabolism etc), other products and/or industrial fermentation, including, but not limited to biofuels from cellulosic biomass, for example, hemicellulosic and cellulosic biomass. Certain embodiments herein provide for modified microorganisms having increased chemical tolerance, or enhanced chemical export, reduced chemical import or enhanced chemical metabolism relative to its wild type and/or control type microorganism. For example, chemicals (e.g. bioproducts or toxic chemicals) contemplated herein can concern low molecular weight organic compounds including, but not limited to, formic acid, acetic acid, citric acid, propionic acid, pyruvic acid, oxalic acid, succinic acid, malonic acid, levulinic acid, formaldehyde, acetaldehyde, and butyraldehyde, furan-2-carbaldehyde (commonly known as furfural), 5-(hydroxymethyl)-2-furaldehyde (commonly known as hydroxymethylfurfural (HMF)) and phenolic aldehydes like benzaldehyde, 4-hydroxy-3-methoxybenzaldehyde (commonly known as vanillin), and (2E)-3-phenylprop-2-enal (commonly known as cinnamaldehyde) or a combination thereof. Some embodiments report use of increased low molecular weight, organic compound (e.g. acetate or aldehyde) tolerance relative to wild type and/or control type organisms of use to increase production of or length of tolerance for byproduct synthesis by the organism.

In one embodiment, a modified microorganism can be a bacteria or yeast or other microorganism capable of producing products from cellulosic biomass. In other embodiments, a modified microorganism can be E. coli. In yet other embodiments, a modified microorganism can be a Zymomonas spp., Saccharomyces spp., and subspecies or other bacterial species capable of producing biofuels from cellulosic biomass. In accordance with these embodiments, some modified microorganisms have an increase in tolerance or coping mechanisms for toxic byproducts of biomass conversion or industrial fermentation as demonstrated by an increase in production of these products and/or growth rate of at least about 1%-about 5%; about 1% to about 10%; about 1% to about 15%; about 1% to about 25%; to over 100% increase compared to a control microorganism population.

Other embodiments report increased copy number of specific genomic regions and chemical-resistant phenotypes of a microorganism to increase toxic chemical tolerance of that microorganism. In accordance with these embodiments, genes can include, but are not limited to, genes that code for proteins involved in construction of outer-cellular components, and genes that code for proteins which are involved in chemical-consuming pathways, chemical exporting and other genes or a combination of genes for toxic chemical inhibition, degradation, export and/or tolerance by the microorganism. Some embodiments report increased copy number of one or more genes in order to increase chemical (e.g. low molecular weight organic compounds for example, acetates, aldehydes etc.) tolerance by a bacterial organism (e.g. E. coli) or yeast. Other embodiments may use increased copy number of one or more genes or gene regions for increased chemical (e.g. low molecular weight organic compounds for example, acetates, aldehydes etc.) tolerance, chemical export, chemical degradation, and/or chemical metabolism in a bacterial organism compared to a control (e.g. pEZseq a cloning vector with no insert) that include, but are not limited to, murC (peptidoglycan biosynthesis (murein synthesis)), fumB (fumarate hydratase (TCA cycle—may consume acetate)), cadA (lysine decarboxylase (acid resistance), yjdL (predicted transporter), cadA-yjdL (two genes next to each other in the genome), argA (N-acetylglutamate synthase (acetyl-CoA consumer)), metH (methionine biosynthesis (methionine supplementation has been shown to confer acetate supplementation)), lpcA, pBTL-1 (a broad host range vector with no insert, lpcA gene in the pBTL-1 vector), nfrB, lgt-thyA operon (lpcA-pBTL1, for example the lpcA gene in the pBTL-1 vector, also known as umpA) and any combination thereof. Some embodiments disclosed herein concern manipulation of certain pathways to modulate tolerance to low molecular weight organic compounds from biomass hydrolysates in a microorganism including, but not limited to, formylTHF biosynthesis I, TCA cycle, glycolysis, arginine biosynthesis, peptidoglycan biosynthesis, lysine biosynthesis, methionine biosynthesis and combinations thereof.

Other vectors contemplated of use in some embodiments herein include, but are not limited to, pEZseq (Lucigen) cloning systems, pSMART cloning systems (Lucigen), pACYCDuet cloning systems (Novagen) pBMT-1, pBMT-2, pBMT-3, pBMT-4, pBMT-5, pBMT-6, pBT-1, pBT-2, pBT-3, pBT-4, pBT-5, pBT-6, pBMTB-1, pBMTB-2, pBMTB-3, pBMTB-4, pBMTB-5, pBMTB-6, pBTB-1, pBTB-2, pBTB-3, pBTB-4, pBTB-5, pBTB-6, pBMTL-1, pBMTL-2, pBMTL-3, pBMTL-4, pBMTL-5, pBMTL-6, pBTL-1, pBTL-2, pBTL-3, pBTL-4, pBTL-5, pBTL-6 or any vector capable of inducing the one or more genes.

In accordance with these embodiments, one or more gene(s) may be increased in expression or copy number to modulate chemical tolerance in a bacterial organism or a combination of genes or gene segments may be used.

In certain embodiments, compositions of use to modulate tolerance to low molecular weight organic compounds found in biomass hydrolysate may include one, two, three, or four, or up to all ten genes disclosed herein alone or in combination with other compositions to modulate tolerance to these compounds. In certain embodiments, modulation of genes in bacterial organisms may include modulation of low molecular weight organic compounds tolerance such as acetate tolerance by a bacterial organism. In accordance with these embodiments, one or more gene(s) may be modulated in a bacterial organism to modulate acetate metabolism including, but not limited to, lpcA, murC, fumB, cadA, yjdL, argA, metH, and any combination thereof.

In other embodiments, modulation of tolerance for low molecular weight organic compounds found in biomass hydrolysates in bacterial organisms may include modulation of one or more pathways or genes associated with these pathways.

In other embodiments, one or more gene(s) may be modulated in a bacterial organism to modulate tolerance of low molecular weight organic compounds having a terminal carbonyl group (e.g. aldehyde), including, but not limited to, lpcA, lgt, nfrB, thyA or combinations thereof or other genes in combination with these genes in order to modulate low molecular weight organic compounds having a terminal carbonyl group production in biomass hydrolysate by a microrganism. In certain embodiments, compositions and methods disclosed herein concern modulating tolerance in a microorganism to furfural and/or hydroxymethylfurfural.

Low molecular weight organic compounds of biomass hydrolysates contemplated in some embodiments disclosed herein can include, but are not limited to, formic acid, acetic acid, citric acid, propionic acid, pyruvic acid, oxalic acid, succinic acid, malonic acid, levulinic acid or any combination thereof. Aldehydes contemplated herein can include, but are not limited to, compounds composed of organic molecules with 10 or less carbons and a formyl group side chain. Carbons of these aldehydes can be oriented in straight-chain conformations or cyclic orientations with one or many side chains. Examples of straight-chain aldehydes include, but are not limited to, formaldehyde, acetaldehyde, and butyraldehyde and combinations thereof. Examples of cyclic aldehydes include, but are not limited to, furan-2-carbaldehyde (commonly known as furfural), 5-(hydroxymethyl)-2-furaldehyde (commonly known as hydroxymethylfurfural) and phenolic aldehydes like benzaldehyde, 4-hydroxy-3-methoxybenzaldehyde (commonly known as vanillin), and (2E)-3-phenylprop-2-enal (commonly known as cinnamaldehyde) and combinations thereof. Modulation of tolerance by microorganisms to any combination of compounds disclosed herein is contemplated.

Some embodiments disclosed herein can include modifying microorganisms to express any genes disclosed herein within the organism and/or cloning or stably integrating additional copies or a predetermined copy number of the disclosed genes into a microorganism for modulating tolerance to a low molecular weight organic compound. Compositions contemplated herein to modulate tolerance to low molecular weight organic compounds may include amino acid supplements.

### DESCRIPTION OF ILLUSTRATIVE EMBODIMENTS

### Definitions

As disclosed herein “low molecular weight organic compounds” can mean low molecular weight organic acids or carbonyl-containing compounds having ten carbons or less. In certain embodiments, these compounds can be linear or cyclic and can include, but are not limited to, formic acid, acetic acid, citric acid, propionic acid, pyruvic acid, oxalic acid, succinic acid, malonic acid, levulinic acid, formaldehyde, acetaldehyde, butyraldehyde, furan-2-carbaldehyde (commonly known as furfural), 5-(hydroxymethyl)-2-furaldehyde (commonly known as hydroxymethylfurfural) and phenolic aldehydes like benzaldehyde, 4-hydroxy-3-methoxybenzaldehyde (commonly known as vanillin), and (2E)-3-phenylprop-2-enal (commonly known as cinnamaldehyde) and combinations thereof.

As disclosed herein “modulate” can mean an increase, a decrease, upregulation, downregulation, an induction or the like.

### BRIEF DESCRIPTION OF THE FIGURES

The following drawings form part of the present specification and are included to further demonstrate certain embodiments of the present invention. The embodiments may be better understood by reference to one or more of these drawings in combination with the detailed description of specific embodiments presented herein.

FIG. 1 represents a schematic of a cellulosic biomass conversion to biofuels.

FIGS. 2A and 2B represent histograms representing clone growth in the presence of A) 1.75 g/L selection or B) 2.5 g/L selection media.

FIGS. 3A and 3B illustrate clone growth under various conditions.

FIG. 4 illustrates bacterial growth under various conditions in the presence or absence of various amino acids.

FIG. 5 illustrates a schematic of hydrolysate inhibitors and their attack on a bacteria.

FIG. 6 represents a blown up region representing a clone fitness mapped over an E. coli genome.

FIGS. 7A-7B represents a schematic of toxic byproduct effects in a microorganism and data collected.

FIGS. 8A-8D represents a blow-up region representing a clone fitness mapped over an E. coli genome and data collected.

FIGS. 9A-9D represents a blow-up region representing a clone fitness mapped over an E. coli genome and data collected.

FIG. 10 represents a histogram plot of certain clones and predominate genes contributing to growth of the organism under certain conditions.

FIG. 11 represents a histogram plot of data obtained under various growth conditions of a microorganism in the presence or absence of targeted gene induction.

FIG. 12 represents a table of various target pathways of some embodiments disclosed herein.

FIG. 13 represents a plot of clone fitness mapped over a bacterial genome, peak size is relative to fitness.

FIG. 14 represents a plot of clone fitness mapped over a bacterial genome, peak size is relative to fitness.

FIG. 15 represents a schematic of gene linkage contemplated regarding some embodiments disclosed herein.

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that concentrations, times, temperature and other details may be modified through routine experimentation. In some cases, well known methods or components have not been included in the description.

In accordance with embodiments of the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Creating biofuels from biomass is an important tool for moving away from dependence on depleting sources of fuels. In addition, enhancing industrial fermentation and/or bioprocessing is needed for increased production and reduced cost. In certain embodiments herein, using cellulosic biomass for generating biofuels in microorganisms is contemplated. Examples of cellulosic biomass include, but are not limited to, using hemicellulosic, lignocellulosic and cellulosic biomass for generating biofuels.

Biofuels derived from lignocellulosic biomass hold promise for making up a significant fraction of this market. One goal is pretreatment to relax the crystalline nature of cellulose for downstream hydrolysis, convert hemicellulose to pentoses, and to remove lignin. Harsh conditions used in pretreatment create a variety of toxic compounds that inhibit the fermentation performance. General classes of inhibitors have been defined: acetic acid release from actylxylan decomposition, furan derivatives from sugar and subsequent degradation, and phenolic compounds derived from lignin. Furan derivatives include, for example, furfural (2-furaldehyde) and 5-hydroxymethylfurfural (HMF), which result from pentose and hexose degradation, respectively. Subsequent degradation of these compounds introduces formic acid (furfural and HMF degradation) and levulinic acid (HMF degradation) into the hydrolysate. Phenolic compounds include, for example, acids, alcohols, aldehydes, and ketones.

Although many fermentative microorganisms exist, in certain embodiments, Escherichia coli, Saccharomyces cerevisiae, and Zymomonas mobilis can serve as industrial biocatalysts for biofuels production. Each microorganism has limitations in native substrate utilization, production capacity, or tolerance. Unlike Saccharomyces cerevisiae or Z. mobilis, E. coli natively ferments both hexose and pentose sugars. Ethanologenic E. coli also has higher tolerance to lignocellulosic inhibitors than its fermentative counterparts.

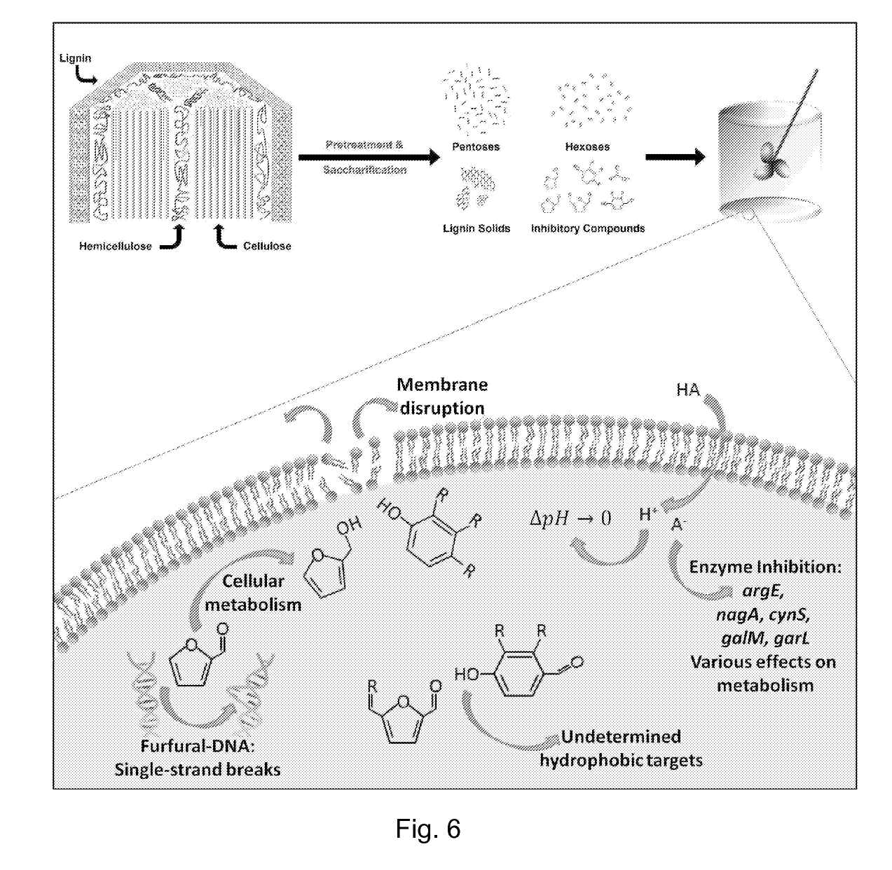

As depicted in FIG. 6, generally accepted categories of antimicrobial activity for inhibitors in lignocellulosic hydrolysate include: (a) compromising the cell membrane, (b) inhibiting essential enzymes, or (c) negative interaction with DNA or RNA. These toxic compounds often act by inhibiting multiple targets. Although efforts are underway to limit the amount and types of inhibitors created during pretreatment, at the present time, economically viable processes still fall short. Regardless of pretreatment optimization, inhibitors such as acetic acid, released directly from hemicelluloses decomposition, remain in the hydrolysate. Thus, a need to engineer more tolerant fermentative microorganisms exists.

### Organic Acids

Organic acids derived from lignocellulosic biomass pretreatment and subsequent saccharification inhibit the growth and metabolism of microorganisms (e.g. E. coli). This, in turn, reduces the yield, titer, and productivity of biofuel fermentation. Various organic acids are created in pretreatment steps: acetic acid is derived from the hydrolysis of acetylxylan, a main component of hemicellulose; others (formic, levulinic, etc.) are a result from degraded sugars.

Acetic acid is usually found at the highest concentration in the hydrolysate. Levels of acetate depend on the type of cellulosic biomass and the pretreatment method. Concentrations can range from 1 to >10 g/L in the hydrolysate. Formic acid, while more toxic to for example, E. coli than acetic acid, is typically present at concentrations much less than that of acetic acid (commonly 10% of acetic acid concentrations). Other toxic weak acids, whose hydrolysate concentrations are rarely reported, are present at an even lower concentration than formic acid.

### Some Modes of Toxicity

Weak organic acids have been shown to primarily inhibit the production of cell mass, but not the fermentation itself. Acetate is a natural fermentation product that is known to accumulate due to “overflow metabolism” and inhibit cell growth. Acetate concentrations as low as 0.5 g/L have been shown to inhibit cell growth by 50% in minimal media.

Weak acids, in the undissociated form can permeate the cell membrane, and once inside, dissociate to release the anion and the proton. These “uncoupling agents” can disrupt the transmembrane pH potential since, effectively, a proton is allowed across the membrane without the creation of ATP. This dissociation of the weak acid inside the cytoplasm is due to the fact the intracellular pH, pHi, is naturally at a pH of ˜7.8, which is much higher than the weak acid's pKa. As these acids dissociate inside the cell, the pHi decreases, which can inhibit growth. External pH has a large affect on the toxicity of the weak acids. E. coli KO11 in LB media with 5.0 g/L acetate reached an ethanol titer twice as fast at an initial pH of 7.0 compared to initial pH of 6.0, and thrice as fast compared to an initial pH of 5.5. When E. coli LY01 was subjected, at a starting pH of 6.0, to acetic, formic, or levulinic acid at the IC50 obtained at a neutral pH, the growth rate decreased to 0, 35, and 10 percent that of control growth. Formic acid may be more toxic due to the fact it has an extraordinarily high permeability through the membrane. This external pH effect is, in part, due to the fact that the acid exists in its undissociated form at higher concentrations, allowing for higher permeation of the cell membrane.

The anion also has an inhibitory effect. The anion accumulates inside the cell, which can affect the cell turgor pressure. Inhibition has been shown to be anion specific. When E. coli inhibition from acetate was compared to benzoate, the same growth rate was observed for differing pHi (7.26 for benzoate and 7.48 for acetate). It has been reported that the toxicity of weak acids depended highly on the hydrophobicity of the acid.

### Furan Derivatives

Furan derivatives are a result of sugar degradation during pretreatment. Furfural and HMF are the primary derivatives appearing in lignocellulosic hydrolysate. Concentrations typically range between 0-6 g/L for each compound. As previously mentioned, levulinic and formic acid are also formed via degradation of these aldehydes. While dilute acid hydrolysis is a common method for pretreatment, acidic conditions are known to cause degradation of a small fraction of the sugar monomers. Hemicellulose is the second most abundant renewable polysaccharide, averaging 25-35% of viable lignocellulosic biomass composition. Therefore, processes that avoid degradation of the C5 and C6 monomers may be important.

Aldehydes are known to have detrimental effects in microorganisms. For example, it was shown that formaldehyde denatures and interacts with polynucleotides. Formaldehyde is also known to cause protein-protein cross-linking. Methylgloxal, a dicarbonyl compound, has been shown to inhibit E. coli growth and protein synthesis at concentrations of 0.07 g/L.

### Some Modes of Toxicity

Furfural has been identified as a key inhibitor in lignocellulosic hydrolysate because it not only is toxic by itself but is also known to act synergistically with other toxins. Hydrophobicity is a marker of an organic compound's toxicity. Highly hydrophobic compounds have been shown to compromise membrane integrity. Intracellular sites are more likely to be the primary inhibition targets of furfural and HMF. In contrast, both 2-furoic acid and furfuryl alcohol have been shown to cause significant membrane leakage. Furfuryl alcohol is less toxic on a concentration basis, by almost an order of magnitude (˜2 g/L for furfural vs. ˜20 g/L furfuryl alcohol) than furfural.

Ethanol production was inhibited in E. coli LYO1 by furfural, suggesting a direct effect on glycotic and/or fermentative enzymes. In the same study, furfural inhibition on aldehyde dehydrogenase (AlDH; EC 1.2.1.5) and the pyruvate dehydrogenase (PDH) complex were investigated and determined to be in this case, more significant than ADH, as evidenced by more than 80% activity reduction in the presence of 0.12 g/L furfural, whereas ADH activity was only inhibited by 60%. This study suggests that furfural may detrimentally affect multiple glycotic enzymes that contribute to central metabolism.

Furfural and HMF have shown cytotoxic characteristics towards both bacteria and yeast. Furfural is a known dietary mutagen and has been under investigation for direct effects on DNA in the past. A series of studies confirmed that furfural-DNA interactions occur. Furfural-treated double-stranded DNA led to single-strand breaks after undergoing in vitro incubation with furfural, primarily at sequence sites with three or more adenine or thymine bases. Later, plasmids treated with furfural were observed to cause either an increase (high furfural concentrations) or decrease (low furfural concentrations) in plasmid size via insertions, duplications, or deletions.

### Phenolic Compounds

Hydrolysates can contain up to 30% lignin content for a variety of feedstocks. Major phenolic compounds have carboxyl, formyl, or hydroxyl group functionalities and arise from degradation of lignin during pretreatment. Ketones can also be released during pretreatment, but are not generally considered as primary inhibitors because they occur at low concentrations (<0.05 g/L) and are also partially or completely removed with various detoxification treatments. Most of the lignin and its derivatives are insoluble; after dilute acid pretreatment of yellow poplar, no more than 15% of the total lignin feedstock content was converted to a soluble species. Concentrations of aromatic monomers after dilute acid washes have been measured between 0-3 g/L and include acids, alcohols, and aldehydes. Due to the number of lignin-derived compounds needing to be analyzed, sequential studies with E. coli have been limited. As such, only the most commonly studied compounds are reviewed in this work.

### Modes of Toxicity

A series of studies comparing aldehydes, acids, and alcohols appearing in hydrolysate were performed with the ethanologenic E. coli LYO1. In general, the degree of toxicity correlated with the compound's octanol/water partition coefficient, log(Poctanol/water), which is a measure of hydrophobicity. In all studies the phenolics were more toxic than aliphatics or furans with the same functional group. This observation that hydrophobicity was related to membrane damage was only true for the alcohols tested, with the exception of hydroquinone. Aromatic acids caused partial membrane leakage while the aromatic aldehydes caused no significant membrane damage. A synergistic binary combination was observed for guaiacol and methylcatechol, but a less than additive combination was observed for vanillyl alcohol and all lignin-derived alcohols tested (catechol, coniferyl, guaiacol, hydroquinone, and methylcatechol). Vanillin, which is a phenolic aldehyde, was found to be a bacteriostatic and membrane active inhibitor, causing partial disruption of K+ gradients in E. coli MC1022. This finding is similar to the effect of methylglyoxal on E. coli. Membrane destabilization was experienced by 29% of the population after treatment with vanillin for one hour at over three times the minimum inhibitory concentration, but restored to 13% when grown overnight. In addition, this study showed that ATP production continues without, significant interruption. In previous reports, membrane damage was found to not contribute significantly to toxicity. From these data, hypotheses have been developed stating that other cellular hydrophobic components may be the primary target for inhibition.

### Modes of Tolerance

From the studies conducted on E. coli LYO1, tolerance to aldehydes benefited from increased inoculum size, suggesting metabolism of the compound. For example, recombinant E. coli are capable of converting aromatic aldehydes to their corresponding acids. Non-lignin derived aromatic acids have also been showed to be metabolized as sole carbon sources, similar to observations of furfural and HMF metabolism. Conversion of an aldehyde to carboxylic acid or alcohol is often beneficial for E. coli due to the reduced toxicity of the functional group.

### Engineering Tolerance

Genomic library selection is a powerful tool that can discover genes or operons that, with increased copy number, confer a desired phenotype. The advent of DNA microarrays has made it easier to identify these beneficial genes. SCALEs (Scalar Analysis of Library Enrichments), and its predecessor PGTM (Parallel Gene Trait Mapping), have used E. coli genomic library selection and microarrays to engineer tolerance to Pine-Sol antibiotic, antimetabolites, 3-hydroxypropionic acid, and naphthol. Genomic selections employing libraries of heterologous genes have also been used to engineer tolerance.

Biofuels production must find cost effective and sustainable feedstocks. The commercial potential of biofuels largely depends on the abundance and cost of the feedstock. As this number grows, commercial processes will necessarily rely more heavily upon lignocellulosic biomass. Much work is still required to improve the efficiency of fermentations of biomass hydrolysate to levels cost competitive with fermentation of pure sugar streams. Emphasis should be placed upon not only further reducing the cost of enzymatic hydrolysis step but also upon better understanding of hydrolysate toxicity mechanisms and methods for engineering tolerance. More specifically, elucidating the modes of action of specific compounds present in hydrolysate may prove critical since the levels of inhibition of various aldehydes and weak acids can vary greatly.

In accordance with these embodiments, increasing tolerance of microorganisms to toxic byproducts of biomass hydrolysis and other processes are disclosed herein. In certain embodiments, increasing growth rates of microorganisms by increasing tolerance to toxic byproducts can be accomplished by genetic manipulation of pathways, for example by modulating one or more genes intricate to the pathway. Some embodiments concern modulating tolerance to toxic byproducts by genetic manipulation of pathways that induce chemical tolerance. In other embodiments, increased tolerance of microorganisms to toxic chemicals can include increased production of recombinant proteins by microorganisms for pharmaceutical applications.

Sustainable production of biofuels will require the efficient utilization of lignocellulosic biomass. One barrier involves the creation of growth-inhibitory compounds chemical pretreatment steps, which ultimately reduce the efficiency of fermentative microbial biocatalysts. The primary toxins include organic acids, furan derivatives, and phenolic compounds. Furan derivatives, which result from degradation of lignocellulosic sugars, have been shown to hinder fermentative enzyme function. Phenolic compounds, formed from lignin, can disrupt membranes and are hypothesized to interfere with the function of intracellular hydrophobic targets.

Aldehydes can be inhibitory compounds to microorganisms, formed during the pretreatment of for example, lignocellulosic biomass. Furan derivatives, such as furfural and hydroxymethylfufural are sugar degradation products, whereas phenolic aldehydes, such as benzylaldehyde and vanillin, are monomers released from lignin. These compounds reduce the efficiency of microbial biocatalysts in biofuel production, recombinant molecule production and biorefining applications.

Acetates can be inhibitory compounds to microorganisms, for example acetates can disturb cellular homeostasis, lower pH, create an accumulation of intracellular anion, retard growth at concentrations as low as 0.5 g/L, reduce cellular glutamate and aspartate pools, as well as other interfering functions. Certain embodiments disclosed herein concern compositions and methods for modulating these and other acetate interferences to microorganisms.

Certain embodiments herein concern modulating one or more pathways capable increasing low molecular weight organic compound tolerance in a microorganism. For example, other embodiments concern modulation of one or more pathways including, but not limited to, formylTHF biosynthesis I, glycolysis, arginine biosynthesis, peptidoglycan biosynthesis, lysine biosynthesis, methionine biosynthesis or combinations thereof.

In certain embodiments, modulation of one or more genes can include modulation of lpcA, a gene capable of modulating ADP-L-glycero-b-D-manno-heptose biosynthesis to increase acetate tolerance of a microorganism. In accordance with these embodiments, a microorganism may have increased tolerance to low molecular weight organic compounds less than ten carbons (e.g acetates and/or aldehydes) having modulated expression or copy number in the microorganism. In other embodiments, modulation of one or more genes can include modulation of cadA capable of modulating one or more of POLYAMSYN pathway, aminopropylcadaverine biosynthesis, and/or lysine degradation to modulate acetate tolerance of a microorganism. Modulation of tolerance to acetates can include increasing a microorganism's tolerance for acetate for example, inducing metabolism, reducing importation or excretion of acetates from the microorganism. In certain embodiments, modulation of one or more genes can include, but are not limited to, modulation of lpcA, murC, fumB, cadA, yjdL, argA, metH or a combination thereof to increase low molecular weight organic compound tolerance of a microorganism.

In certain embodiments, modulation of a gene can include modulation of lpcA (e.g. for aldehydes and acids tolerance induction), lgt (e.g for aldehyde tolerance), nfrB (e.g. for aldehyde tolerance), and thyA (e.g. for aldehyde tolerance). Two of these genes, lgt and thyA, naturally occur within the E. coli chromosome as an operon, or a single transcriptional unit and may be induced simultaneously. Three of these aldehyde tolerance genes relate to membrane-localized modifications or proteins. In certain embodiments, one or more of these membrane localized modifications may be targeted to modulate tolerance in a microorganism to low molecular weight organic compounds of biomass hydrolysis. For example, gene lpcA is the first committed step in lipopolysaccharide core biosynthesis. The substrate in which it catalyzes an isomerization reaction is D-sedoheptulose-7-phosphate. The second membrane-related gene, lgt, is involved in prolipoprotein modification through its protein product, lgt, which is an essential membrane protein. The third gene, nfrB, is an inner membrane subunit that has been attributed to N4 bacteriophage adsorption. thyA, is involved directly in de novo DNA synthesis. In addition, this DNA synthesis step is coupled with the process of producing folate derivatives within the cell. Folate derivatives are used for multiple reactions within the cell, but one specific derivative, tetrahydrofolate, THF, is also associated with a gene found during the acid selection, metH. This gene is found associated with formylTHF biosynthesis I as is meth, discussed previously.

Other embodiments report increased copy number of specific genomic regions and chemical-resistant (e.g. aldehyde-resistant, acetate-resistant) phenotypes. In accordance with these embodiments, genes can include, but are not limited to, genes that code for proteins involved in construction of outer-cellular components, and genes that code for proteins which are involved in toxic chemical-consuming pathways and other genes or a combination of genes for toxic chemical inhibition and tolerance. Some embodiments report increased copy number of one or more genes in order to increase chemical tolerance by an organism (e.g. acetate, aldehyde, in a bacterial organism). Other embodiments may use increased copy number of one or more genes or gene regions compared to a control (e.g. pEZseq a cloning vector with no insert) that include, but are not limited to, murC (peptidoglycan biosynthesis (murein synthesis)), fumB (fumarate hydratase (TCA cycle—may consume acetate)), cadA (lysine decarboxylase (acid resistance), yjdL (predicted transporter), cadA-yjdL (two genes next to each other in the genome), argA (N-acetylglutamate synthase (acetyl-CoA consumer)), metH (methionine biosynthesis (methionine supplementation has been shown to confer acetate supplementation)), acs (acetyl-CoA synthetase (acetate consumer)), insI (operon, ins(N, I, O)-1-DNA recombination), lpcA, pBTL-1 (a broad host range vector with no insert), and lpcA-pBTL1 (the lpcA gene in the pBTL-1 vector) and a combination thereof. In accordance with these embodiments, one gene may be increased in expression or copy number to increase acetate tolerance of a bacterial organism or a combination of genes or gene segments may be used. Yet other embodiments report modulation of one or more genes or genetic regions to increase acetate tolerance of a bacterial organism including, but not limited to, lpcA, murC, fumB, cadA, yjdL, argA, metH, lgt-thyA operon, thyA, nfrB or combinations of two or more thereof.

Vectors contemplated of use in some embodiments herein can include, but are not limited to, pEZseq (Lucigen) cloning systems, pSMART cloning systems (Lucigen), pACYCDuet cloning systems (Novagen) pBMT-1, pBMT-2, pBMT-3, pBMT-4, pBMT-5, pBMT-6, pBT-1, pBT-2, pBT-3, pBT-4, pBT-5, pBT-6, pBMTB-1, pBMTB-2, pBMTB-3, pBMTB-4, pBMTB-5, pBMTB-6, pBTB-1, pBTB-2, pBTB-3, pBTB-4, pBTB-5, pBTB-6, pBMTL-1, pBMTL-2, pBMTL-3, pBMTL-4, pBMTL-5, pBMTL-6, pBTL-1, pBTL-2, pBTL-3, pBTL-4, pBTL-5, pBTL-6 or any vector capable of inducing the one or more genes.

Some embodiments concern modulating tolerance to toxic byproducts by genetic manipulation of pathways that induce aldehyde tolerance. In other embodiments, modulating tolerance of microorganisms to toxic aldehydes may be accomplished by increasing export of, metabolism of, or hardiness of a microorganism to toxic aldehydes. In accordance with these embodiments, a microorganism having at least one of these traits may be used for example, to produce altered amounts of recombinant proteins for pharmaceutical applications, biofuels or other products from microorganisms. In certain embodiments, modulation of one or more genes can include modulation of lpcA, lgt, nfrB, thyA or a combination thereof (e.g. for modulation of aldehyde tolerance).

Certain embodiments herein concern modulating export of toxic aldehydes by a microorganism to maintain predetermined detrimental levels of low molecular weight organic compounds (e.g. acetates, aldehydes). For example, some embodiments herein report maintaining an intracellular level approximately in equilibrium with extracellular concentrations, about 0.1 to about 5 g/L of furfural by the microorganism. Other embodiments concern modulation of metabolism of toxic aldehydes in a microorganism to increase toxic aldehyde tolerance of the microorganism.

Yet other embodiments concern modulation of importing toxic aldehydes by the microorganism. For example, genetic manipulation of the microorganism to reduce uptake of toxic low molecular weight organic compounds is contemplated. In one embodiment, a genome-wide plasmid-based library selection was performed on solid minimal media supplemented with furfural or other toxic aldehyde to identify genetic elements that confer increased tolerance to furfural (see for example, Table 1). In other embodiments, modulated doubling times and cell densities can be observed for clones expressing one or more of these genes exposed to toxic aldehydes (e.g. furfural solutions), compared to a wild-type control.

lgt-thyA operon lgt has been shown to be involved in the lipid modification of prolipoprotein by transferring the sn-1,2-diacylglyceryl group from phosphatidylglycerol to the sulfhydryl group of the N-terminal cysteine. Sequence analysis, mutation, and expression studies show that lgt and the gene immediately downstream, thyA, form an operon. Mutation, complimentation, and membrane separation experiments show that Lgt is an essential membrane protein. Sequence analysis, chemical inactivation, mutation, and complementation studies indicate that His-103 is essential for the activity of the enzyme, and Tyr-235 and His-196 also have significant roles in its function. thyA is involved in DNA synthesis, namely thymidylate synthase, conversion of dUMP to dTMP is the main pathway of de novo dTMP synthesis in the cell. In addition, this DNA synthesis step is coupled with the process of producing folate derivatives within the cell. nfrB is an inner membrane subunit, associated with bacteriophage adsorption.

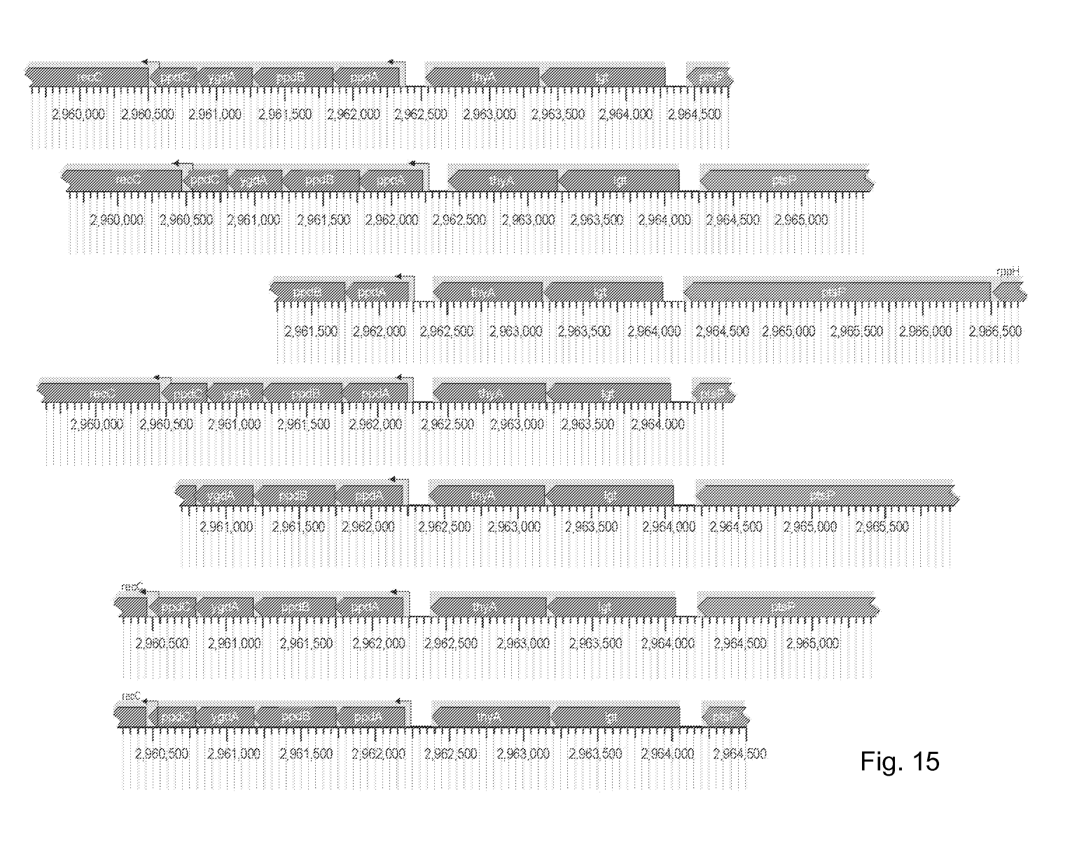

Modulation as disclosed herein may mean inducing or inhibiting, for example, expression or activity of one or more of genes or gene clusters outlined (see for example FIG. 15) above and/or up-regulating or down-regulating the expression of a genetic component identified herein to increase or decrease toxic aldehyde tolerance of a microorganism.

Certain embodiments concern biorefining, biomass (crops, trees, grasses, crop residues, forest residues, etc) and using biological conversion, fermentation, chemical conversion and catalysis to generate and use compounds. These compounds can then subsequently be converted to valuable derivative chemicals. However, of low molecular weight organic compounds may be toxic by nature and thus inhibitory to the production organisms at relatively low levels. In order to optimize production, engineering tolerance to the organic acid may be a factor. This can be accomplished by supplying exogenous molecules to enhance tolerance or to inhibit expression of a non-permissive molecule thereby permitting increased levels of conversion. Since commodity chemicals exist in a competitive environment, optimization might be necessary for the economic feasibility of processing biomass into biofuels or industrial. Therefore, compositions and methods disclosed herein are directed toward identifying bacterial strains and genetic regions within molecules that increase tolerance to toxic aldehydes for use in bioproduction products and systems.

In various embodiments, growth can be enhanced by identifying genes that with increased or decreased expression can increase the tolerance to toxic of low molecular weight organic compounds. Genetic screens, used to detect individual compounds, often proceed one cell at a time. Selections are tied to viability in a specific environment. Therefore, in one embodiment, bacterial organisms that demonstrate increased growth or tolerance for toxic aldehydes may be selected for and the genetic region that affects growth, production and/or tolerance identified. In one embodiment, modulation of genes disclosed herein demonstrate increased growth or tolerance for toxic of low molecular weight organic compound production.

Certain embodiments concern a gene region that is capable of enhancing tolerance of toxic aldehyde or acetate production and/or increasing production of biomass conversion of a microorganism. In accordance with these embodiments, expression of certain molecules within particular genomic regions may be capable of tolerance of production of toxic aldehydes. For example, strains already engineered to convert biomass such as cellulosic biomass can be modified using genetic engineering technologies disclosed herein to a) increase the conversion of biomass and/or b) increase tolerance of the strain to toxic of low molecular weight organic compounds. In addition, these methods may be used in conjunction with the SCALEs technology (Provisional Application No. 60/611,377 filed Sep. 20, 2004 and U.S. patent application Ser. No. 11/231,018 filed Sep. 20, 2005, both entitled: “Mixed-Library Parallel Gene Mapping Quantitation Microarray Technique for Genome Wide Identification of Trait Conferring Genes” incorporated herein by reference in their entirety), for genetic alterations of organisms and for genetic selection strategies.

Genetic manipulation of microorganisms can be used to make desired genetic changes that can result in desired phenotypes and can be accomplished through numerous techniques including but not limited to using a i) vector to introduce new genetic material, ii) genetic insertion, disruption or removal of existing genetic material, as well as, iii) mutation of genetic material or any combinations of i, ii, and iii, that results in desired genetic changes with desired phenotypic changes. A vector can be defined as any genetic element used to introduce new genetic material into an organism and can include, but is not limited to, a plasmid of any copy number, an intergratable element that integrate at any copy into the genome, a virus, phage or phagemid. Genetic insertions, disruptions or removals can be defined as inserting a new genetic element into the genome, disruption transcription or normal regulatory function via insertion that can affect larger regions of the genome in addition to those at the site of insertion, and the deletion or removal of a region of the genome. These can be done with techniques including, but not limited to, directed knock outs or mutations, gene replacements, transposons, random mutagenesis or a combination thereof. Mutations can be directed or random, utilizing any techniques requiring vectors, insertions, disruptions or removals, in addition to those including, but not limited to, error prone or directed mutagenesis through PCR, mutator strains, and random mutagenesis.

Analysis of Library Enrichment (SCALEs), a new high-resolution, genome-wide approach that can be used to monitor enrichment and dilution of individual clones within a genomic-library population, was recently developed. This method includes creation of representative genomic libraries with varying insert size, growth of clones in selective environments, interrogation of the selected population using microarrays, and a mathematical multi-scale analysis to identify the gene(s) for which increased copy number improves overall fitness. In certain embodiments, selections were performed on solid media supplemented with an aldehyde (furfural). Surviving colonies were used to inoculate clones cultures, from which plasmid DNA was extracted and sequenced to reveal the library insert sequence.

The SCALEs method may be employed to develop the technique of directed strain selection for relevant toxic aldehyde and acetate tolerance phenotypes. Initial selections carried out in continuous culture (e.g. E. coli, Zymomonas spp. and subspecies) with different concentrations of toxic of low molecular weight organic compounds revealed various tolerant phenotypes. In certain methods, growth rates were increased in various cultures reflecting an increase in toxic of low molecular weight organic compounds tolerance due to one or more of modulation of transporters, amino acid production and various energy production.

Genomic libraries are a common methods for performing plasmid-based overexpression selections. Individual clones conferring a desirable phenotype (e.g., of low molecular weight organic compound tolerance) within a genomic-library population can be selected for using genomic libraries. This method includes creation of representative genomic libraries with varying insert size, growth of clones in selective environments, and sequencing surviving cells.

Organisms contemplated of use herein include but are not limited to any bacterial culture capable of producing a product, sensitive to increased production of toxic of low molecular weight organic compounds, for example Escherichia coli, Pseudomonas putida, Psedumonas aeruginosa, Zymomonas spp. and subspecies (e.g. Zymomonas mobilis), Clostridia acetobutylicum, Clostridia beijerinckii, Sacchoromyces cerevisiae, Pichia pastoris or combinations thereof are contemplated.

Traditional methods to engineer cells has relied upon multiple rounds of random mutation and selection of those cells that show improved traits. In addition to being laborious these methods cause mutations that are largely ineffective as well as produce cells that appear “sick”. Traditional methods also fail to identify those mutations that are beneficial toward conferring the cellular trait. To address these limitations, pools of synthetic DNA containing molecular barcode tags, regulatory elements and gene homology regions that allow precise insertion upstream of ˜4000 genes in E. coli. The pool of synthetic DNA is then transformed into E. coli and chromosomal insertion is catalyzed by the bacteriophage λ-Red proteins, termed “Recombineering”. Insertion of synthetic regulatory elements increase or decrease downstream gene expression. Insertion of barcode tags allows genome-wide identification of mutants in a complex population on a universal microarray. Beneficial mutations can be accumulated by successive rounds of selection and insertion. Multiplex DNA synthesis and multiplex recombineering are currently being optimized.

### Nucleic Acids

In various embodiments, isolated nucleic acids may be used as test compounds for increasing toxic of low molecular weight organic compounds tolerance in a microorganism. The isolated nucleic acid may be derived from genomic RNA or complementary DNA (cDNA). In other embodiments, isolated nucleic acids, such as chemically or enzymatically synthesized DNA, may be of use for capture probes, primers and/or labeled detection oligonucleotides.

A “nucleic acid” includes single-stranded and double-stranded molecules, as well as DNA, RNA, chemically modified nucleic acids and nucleic acid analogs. It is contemplated that a nucleic acid may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000 or greater nucleotide residues in length, up to a full length protein encoding or regulatory genetic element.

### Construction of Nucleic Acids

Isolated nucleic acids may be made by any method known in the art, for example using standard recombinant methods, synthetic techniques, or combinations thereof. In some embodiments, the nucleic acids may be cloned, amplified, or otherwise constructed.

The nucleic acids may conveniently comprise sequences in addition to a portion of a lysine riboswitch. For example, a multi-cloning site comprising one or more endonuclease restriction sites may be added. A nucleic acid may be attached to a vector, adapter, or linker for cloning of a nucleic acid. Additional sequences may be added to such cloning and sequences to optimize their function, to aid in isolation of the nucleic acid, or to improve the introduction of the nucleic acid into a cell. Use of cloning vectors, expression vectors, adapters, and linkers is well known in the art.

### Recombinant Methods for Constructing Nucleic Acids

Isolated nucleic acids may be obtained from bacterial or other sources using any number of cloning methodologies known in the art. In some embodiments, oligonucleotide probes which selectively hybridize, under stringent conditions, to the nucleic acids of a bacterial organism. Methods for construction of nucleic acid libraries are known and any such known methods may be used.

### Nucleic Acid Screening and Isolation

Bacterial RNA or cDNA may be screened for the presence of an identified genetic element of interest using a probe based upon one or more sequences. Various degrees of stringency of hybridization may be employed in the assay. As the conditions for hybridization become more stringent, there must be a greater degree of complementarity between the probe and the target for duplex formation to occur. The degree of stringency may be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium. In certain embodiments, the degree of complementarity can optimally be about 100 percent; but in other embodiments, sequence variations in the RNA may result in <100% complementarity, <90% complimentarity probes, <80% complimentarity probes, <70% complimentarity probes or lower depending upon the conditions. In certain examples, primers may be compensated for by reducing the stringency of the hybridization and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. Other exemplary conditions are disclosed in the following Examples. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids may be completely complementary to a target sequence or may exhibit one or more mismatches.

### Nucleic Acid Amplification

Nucleic acids of interest may also be amplified using a variety of known amplification techniques. For instance, polymerase chain reaction (PCR) technology may be used to amplify target sequences directly from bacterial RNA or cDNA. PCR and other in vitro amplification methods may also be useful, for example, to clone nucleic acid sequences, to make nucleic acids to use as probes for detecting the presence of a target nucleic acid in samples, for nucleic acid sequencing, or for other purposes.

### Synthetic Methods for Constructing Nucleic Acids

Isolated nucleic acids may be prepared by direct chemical synthesis by methods such as the phosphotriester method, or using an automated synthesizer. Chemical synthesis generally produces a single stranded oligonucleotide. This may be converted into double stranded DNA by hybridization with a complementary sequence or by polymerization with a DNA polymerase using the single strand as a template. While chemical synthesis of DNA is best employed for sequences of about 100 bases or less, longer sequences may be obtained by the ligation of shorter sequences.

### Kits

In certain embodiments, a kit contemplated herein may include means for supplying a microorganism with increased ability to tolerate toxic chemical (e.g. of low molecular weight organic compounds, acetates, aldehydes) production and/or modulate byproduct production. Contemplated herein are means for modulating one or more genes or gene clusters capable of increasing toxic chemical tolerance of a microorganism. Some embodiments report kits having one or more compositions for increasing copy number of genes or gene regions of bacterial (e.g. E. coli) cultures disclosed herein that increase low molecular weight organic compounds tolerance of the bacterial culture. Other kits may include compositions having one or more gene or gene regions for transfecting a bacterial culture to increase acetate tolerance of the bacterial culture. The kits may include a container means. Any of the kits will generally include at least one vial, test tube, flask, bottle, syringe or other container means, into which the testing agent, may be preferably and/or suitably aliquoted. Kits herein may also include a means for comparing the results such as a suitable control sample such as a positive and/or negative control. In yet other embodiments, kits may include one or more vector for inducing one or more genes selected from the group consisting of lpcA, lgt, nfrB, thyA, murC, fumB, cadA, yjdL, argA, metH and a combination thereof.

### EXAMPLES

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

### Example 1

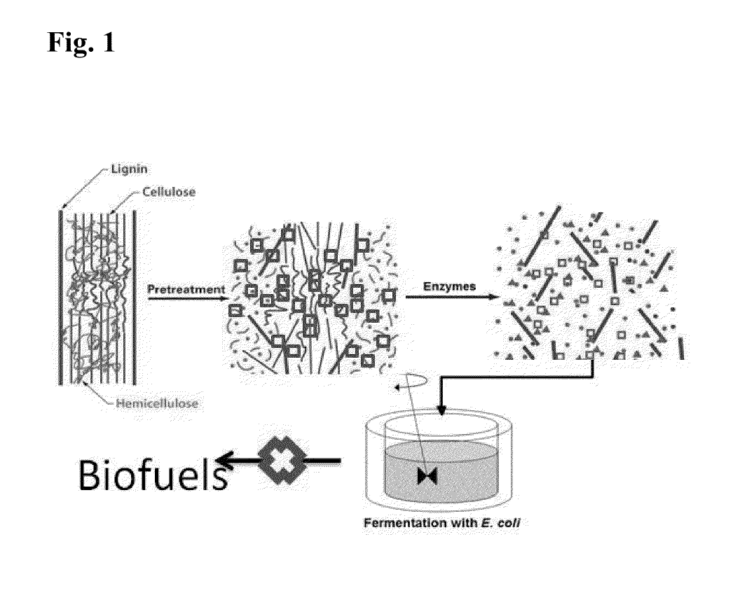

FIG. 1 represents a schematic of a cellulosic biomass conversion to biofuels.

Hydrolysate inhibitors. Lignocellulosic biomass is processed into component sugars, lignin solids, and inhibitory compounds. These inhibitors can affect microbial growth in various ways, including DNA mutation, membrane disruption, intracellular pH drop, and other cellular targets.

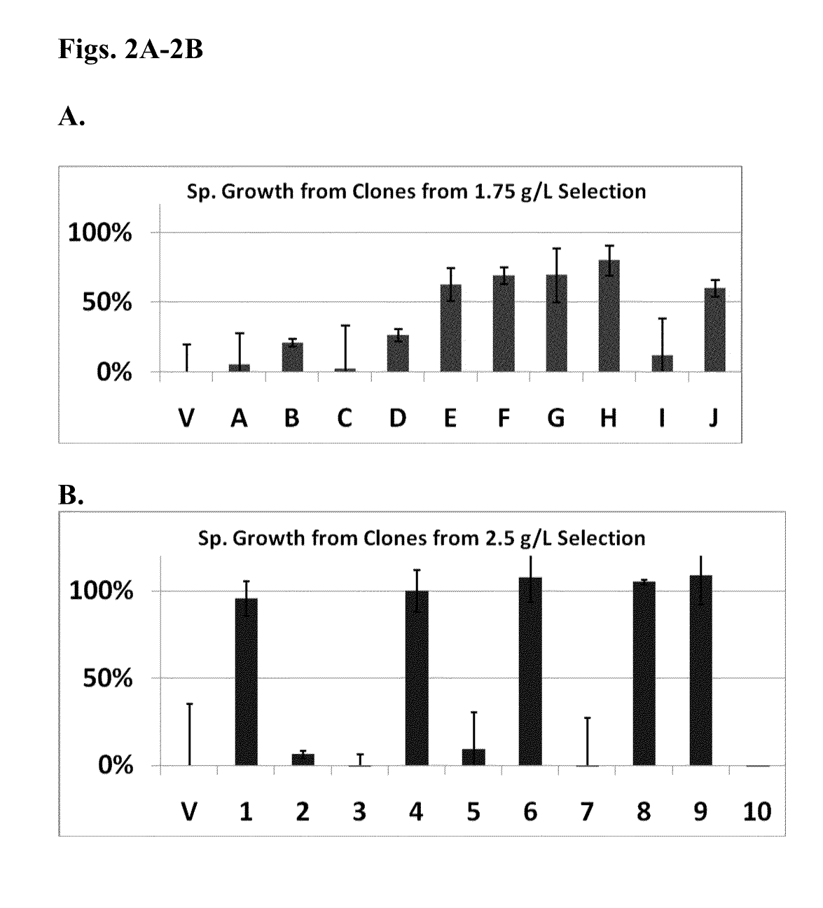

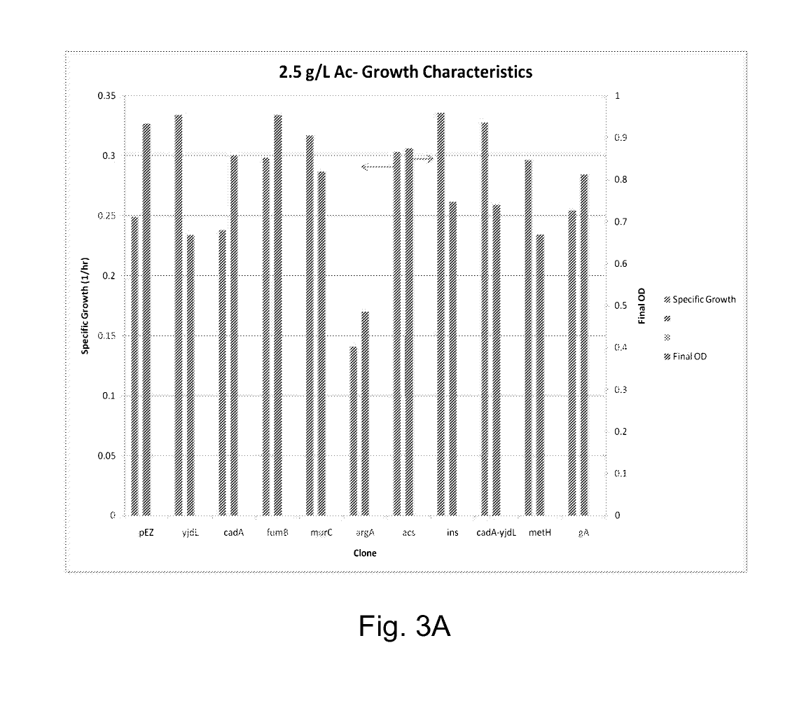

Confirmation of clones found after selection. In one example, after selection, 10 clones were randomly picked from LB Agar+Carb100 plates. All clones plus control were tested for specific growth rate at the specified concentration. Specific growth rate was plotted as percent increase or decrease compared to control specific growth. In one exemplary method bacterial cultures were exposed to two different concentrations of acetate and clones were selected for increased growth in the presence of the increased acetate. These exemplary methods are represented in FIGS. 2A and 2B. FIGS. 2A and 2B represent histograms of clone growth in the presence of A) Clones E, F, G, H, and J in the 1.75 g/L study contained the lpcA gene, and B) Clones 1, 4, 6, 8, and 9 in the 2.5 g/L study contained the lpcA gene. One method used herein was the Scales method previously described.

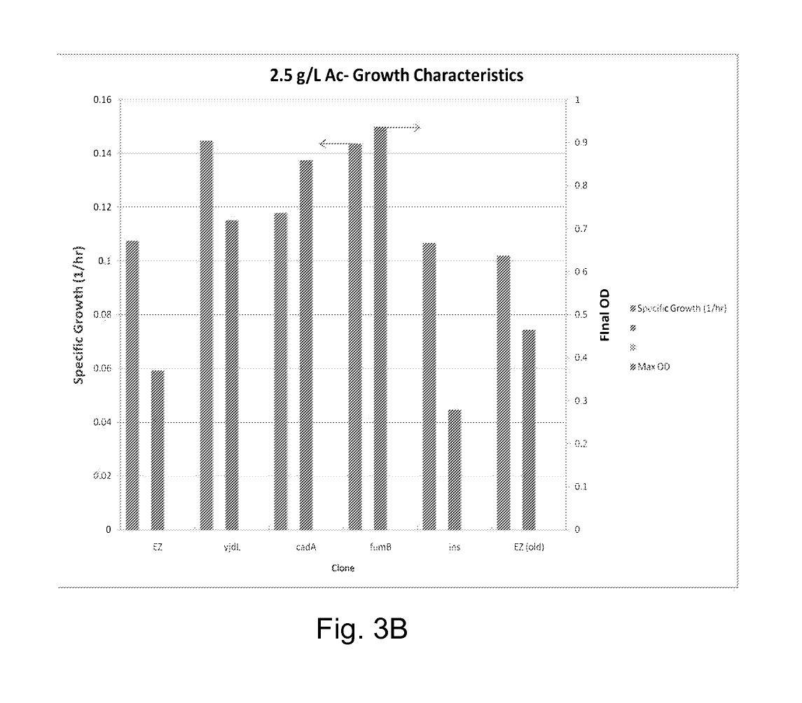

FIGS. 3A and 3B Confirmation of Clone Growth. Overnight cultures were prepared from freezer stocks. Stationary phase overnight cultures were used for a 2.5% inoculation of 5 mL MOPS minimal medium plus carbenicillin. The OD600 was monitored until the culture reached an OD600=0.2. Growth curves were constructed by introducing a 5% inoculation into 5 mL MOPS minimal medium plus carbenicillin supplemented with prepared acetic acid solution to a final concentration 2.5 g/L in a 15 mL centrifuge tube or with 50 mL of media in a 250 mL shake flask. Stock acetic acid solution was prepared by titrating 5 mL of an HPLC-grade 50% acetic acid solution (Fluka) on ice with 10 M KOH to neutral pH. Cultures were incubated at 37° C. and were shaken at 225 rpm. OD600 was monitored over the course of exponential growth and final measurements were taken after 24 hours. Specific growth rate was calculated by linear regression on the natural logarithm of the exponential phase OD600 over time. For each clone, 3A and 3 B, the left bar depicts the specific growth of the clone (1/hr). The right bar depicts the final OD of the clone (proportional to the highest population achieved).

### Example 2

### Method for Growth Rate Testing

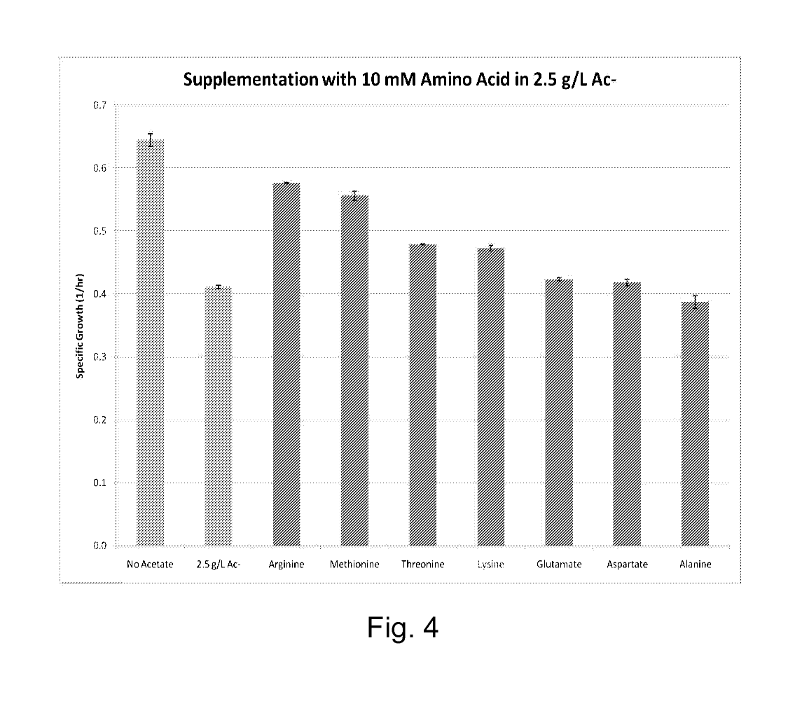

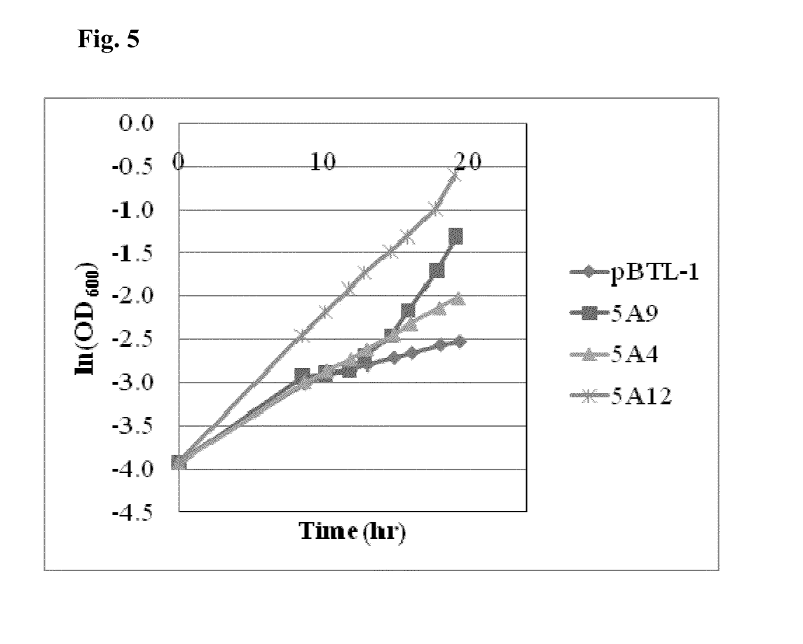

FIG. 4. In another exemplary method, amino acid supplementation of cultures was observed and illustrated by histogram plot. Overnight cultures were prepared from freezer stocks. Stationary phase overnight cultures were used for a 2.5% inoculation of 5 mL MOPS minimal medium plus carbenicillin. The OD600 was monitored until the culture reached an OD600=0.2. Growth curves were constructed by introducing a 5% inoculation into 5 mL MOPS minimal medium plus carbenicillin supplemented with prepared acetic acid solution to a final concentration 2.5 g/L in a 15 mL centrifuge tube or with 50 mL of media in a 250 mL shake flask. Stock acetic acid solution was prepared by titrating 5 mL of an HPLC-grade 50% acetic acid solution (Fluka) on ice with 10 M KOH to neutral pH. Amino acid supplementation was done by preparing stock solutions of amino acids and supplementing the media to a final concentration of 10 mM. Cultures were incubated at 37° C. and were shaken at 225 rpm. OD600 was monitored over the course of exponential growth and final measurements were taken after 24 hours. Specific growth rate was calculated by linear regression on the natural logarithm of the exponential phase OD600 over time. Supplementation of arginine and methionine give the greatest increase in growth rate.

### Example 3

Confirmation of pilot SCALEs selection on furfural tolerance by log-transformation of growth curve data. Clone isolates from the pilot SCALEs furfural selection were grown in 5 ml MOPS minimal media, 100 μg carbenicillin/ml, and 1 g furfural/1 supplemented in 15 ml conical tubes at 37° C. The optical density of 1 ml culture was measured with a spectrophotometer at 600 nm. A natural log transformation of the data were then calculated and are shown here in order to observe relevant lag times and specific growth. The blank vector, pBTL-1, served as the control. Clone 5A4 contains the operon Igt-thyA, Clone 5A9 contains the gene lpcA, and Clone 5A12 contains the gene nfrB. It should be noted that these clones are direct isolates from the SCALEs pilot selection and therefore are products of library construction (e.g., fragments or whole genes are also included within the vector's insert DNA). These clones serve as the parent clones from which genes of interest could be subcloned.

This data supports that there are multiple genes that can be attributed to conferring furfural tolerance. Both Clones 5A12 (nfrB parent clone) and 5A4 (lgt-thyA parent clone) represent cultures that do not undergo the characteristic lag phase experienced by pBTL-1 control cells. Clone 5A9 (lpcA parent clone) undergoes a lag phase, but then in followed by a significantly increased specific growth after about 12 hours.

### Example 4

Hydrolysate inhibitors. Lignocellulosic biomass is processed into component sugars, lignin solids, and inhibitory compounds. These inhibitors can affect microbial, growth in various ways, including DNA mutation, membrane disruption, intracellular pH drop, and other cellular targets. FIG. 6 represents a schematic of some examples of sites of low molecular weight organic compound attack on a microorganism.

### Example 5

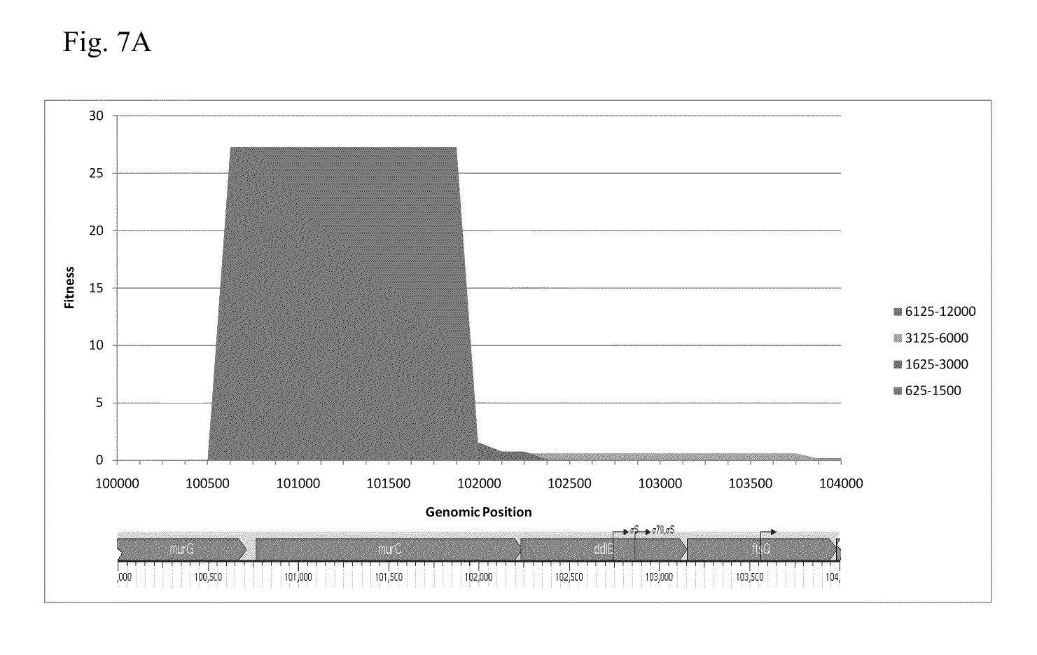

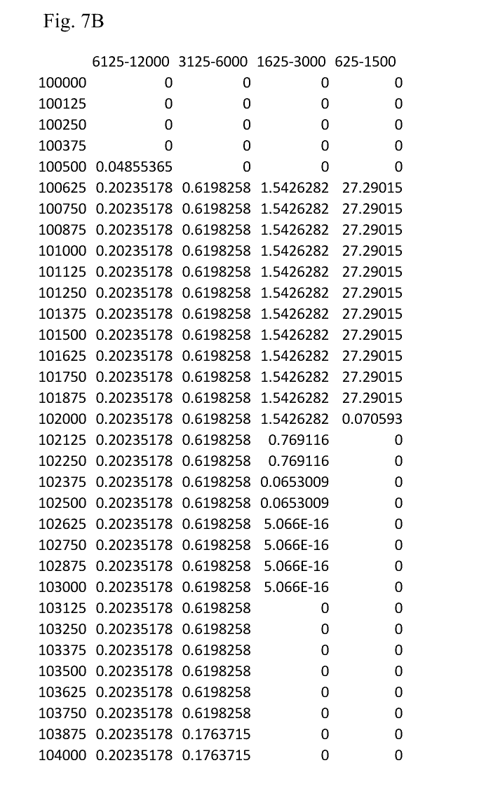

In some exemplary methods, blow-up regions are represented in FIGS. 7A and 7B. Clone fitness mapped over E. coli genome. Peak size is relative to fitness. Colors denote the size of the clones: Blue 1 kb, Red 2 kb, Green 4 kb, and Purple 8 kb (shown in black and white). In detail, fitness value plotted over genomic position. Here is illustrated gene murC and surrounding area from the 1.75 g/L SCALEs selection and analysis.

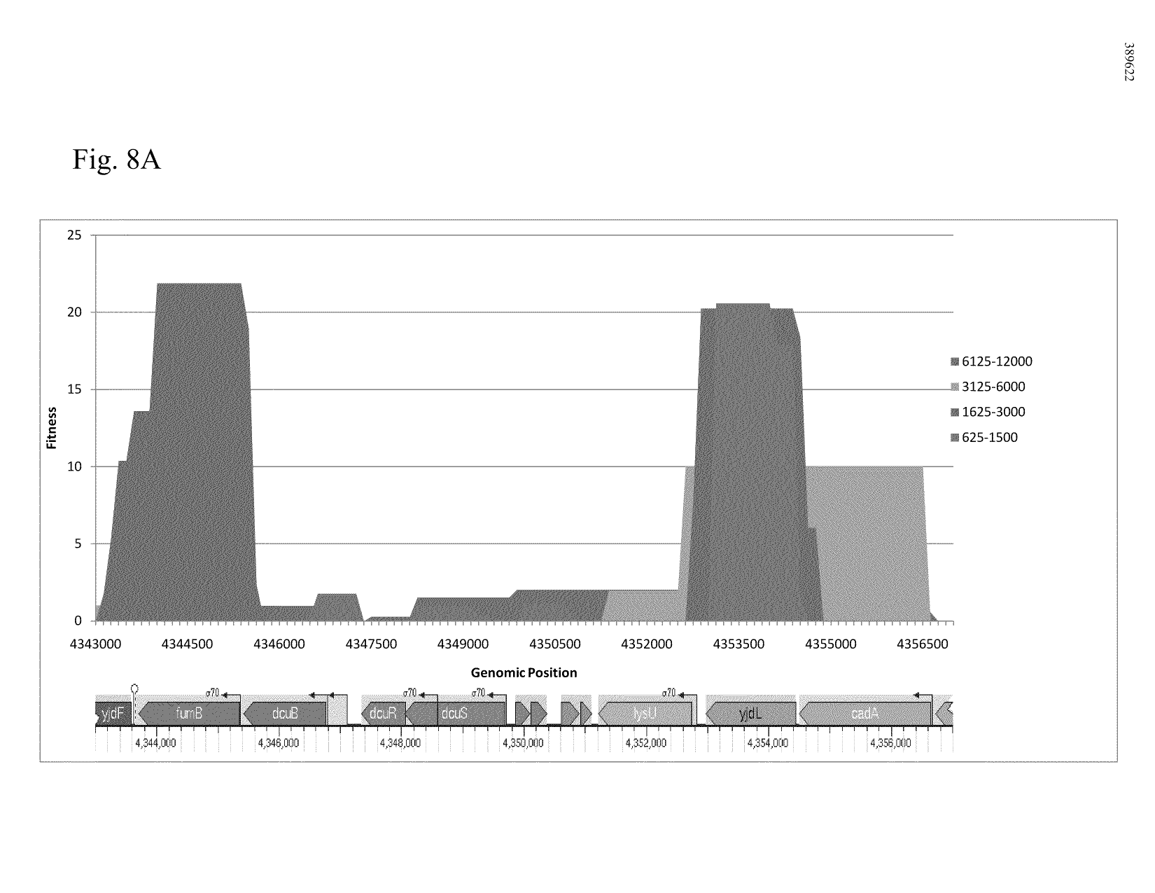

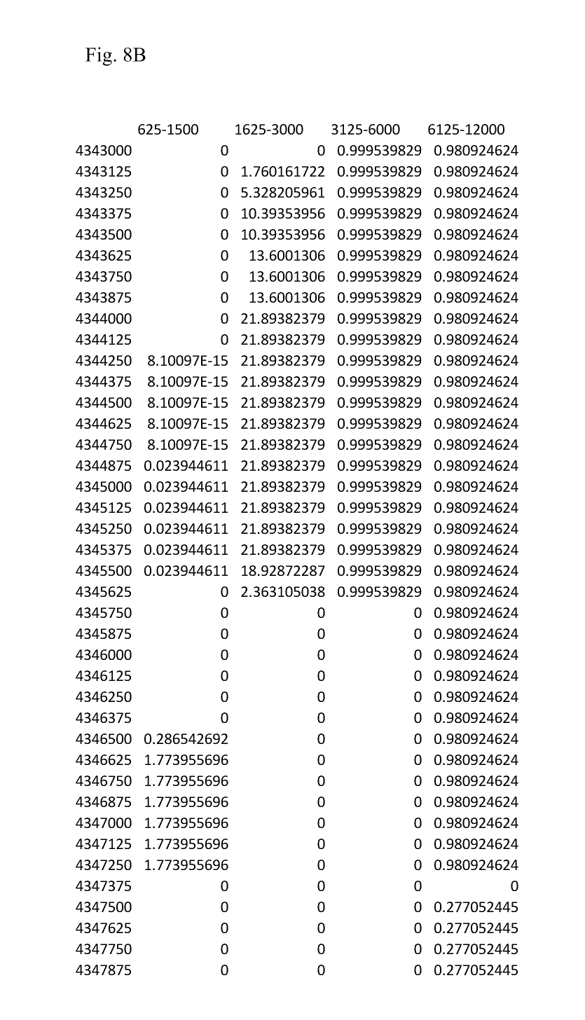

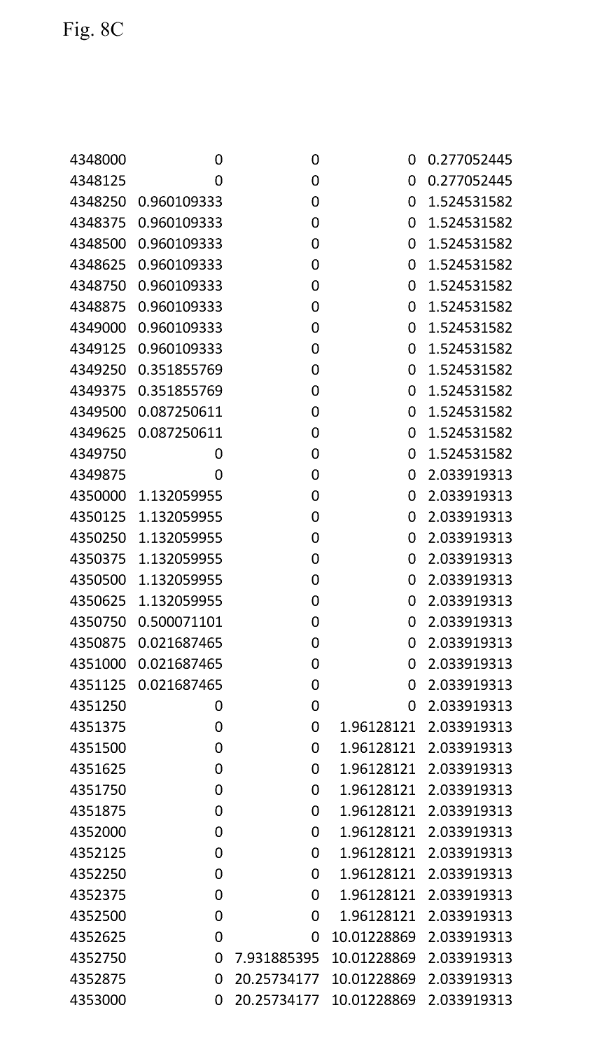

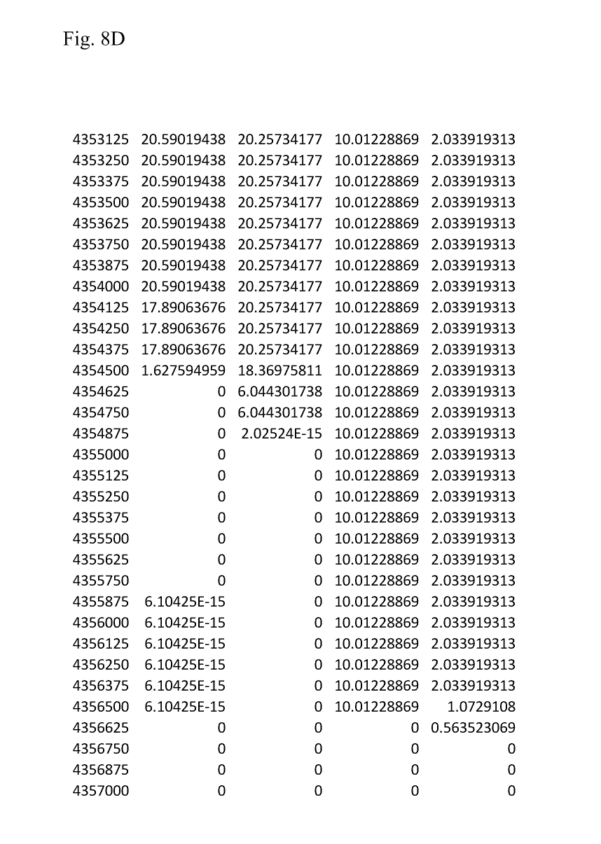

FIGS. 8A-8D. Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrated a region of the genome that contains the genes yjdL, cadA, and fumB from the 1.75 g/L SCALEs selection and analysis.

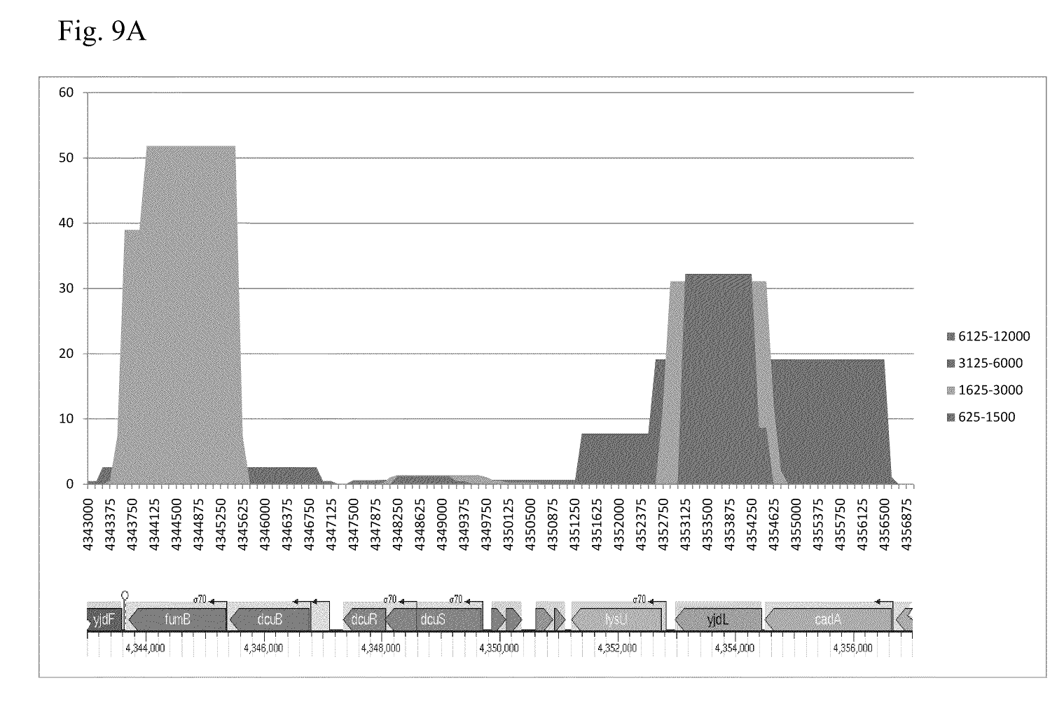

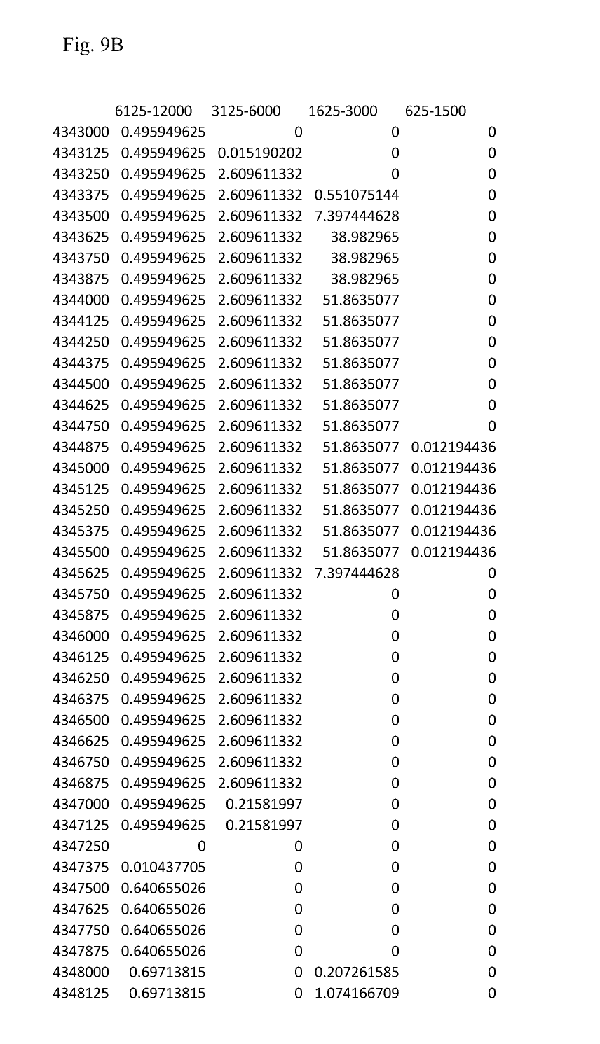

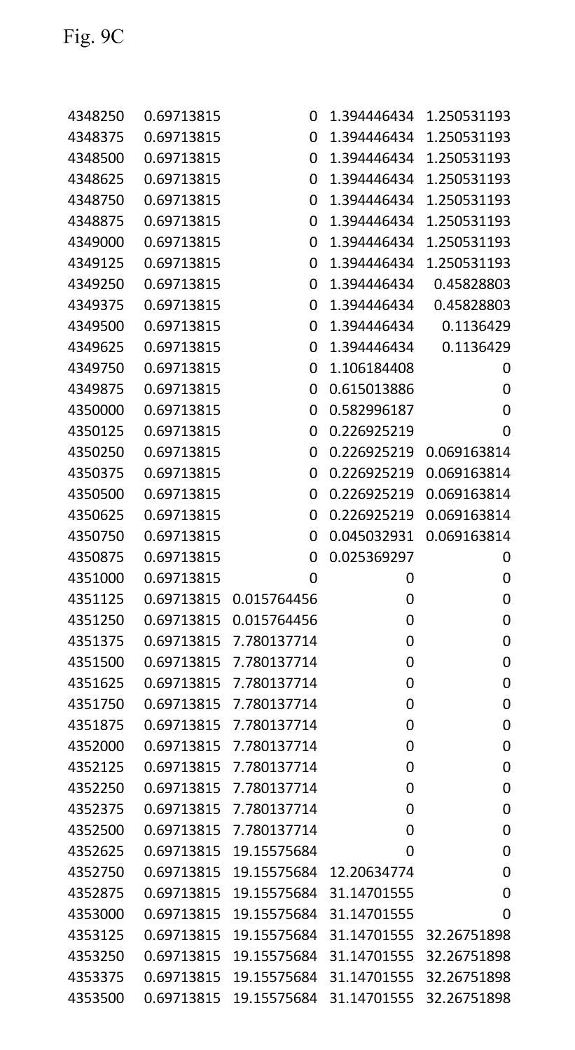

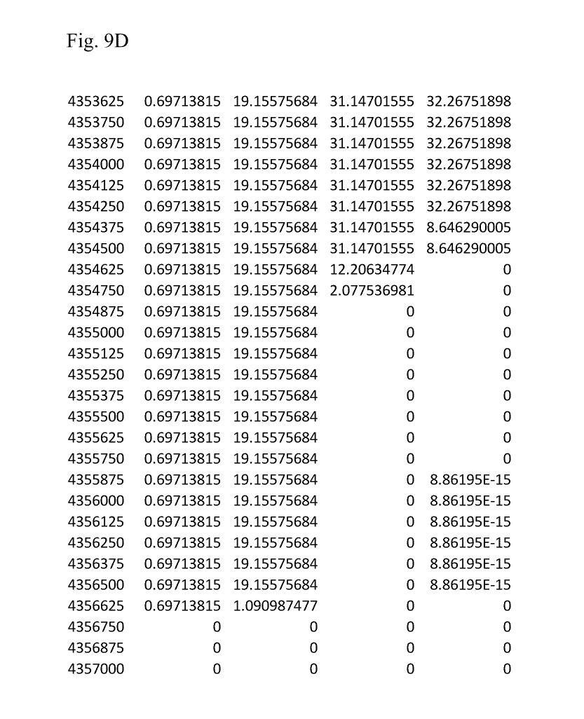

FIGS. 9A-9D. Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrated a region of the genome that contains the genes yjdL, cadA, and fumB from the 2.5 g/L SCALEs selection and analysis.

### Example 6

A pilot SCALEs selection was run on furfural tolerance. A SCALEs library was constructed in the pBTL-1 expression vector system. Cells were transformed with library plasmids and plated onto MOPS minimal media plates, supplemented with varying levels, 1-5 g/l, of furfural. Plates were incubated at 37° C. until colonies appeared, up to four days. Colonies were randomly selected to test for confirmation of improved tolerance phenotype.

Then, a SCALEs selection was performed in a similar fashion as the pilot selection, with the addition of microarray analysis, as prescribed by the SCALEs method. The expression vector system used for the SCALEs furfural selection was pSMART-LCK (Lucigen). Fitness values were calculated based on microarray analysis.

Certain experiments described herein were performed in E. coli BW25113 recA-, with the kanamycin resistance gene removed.

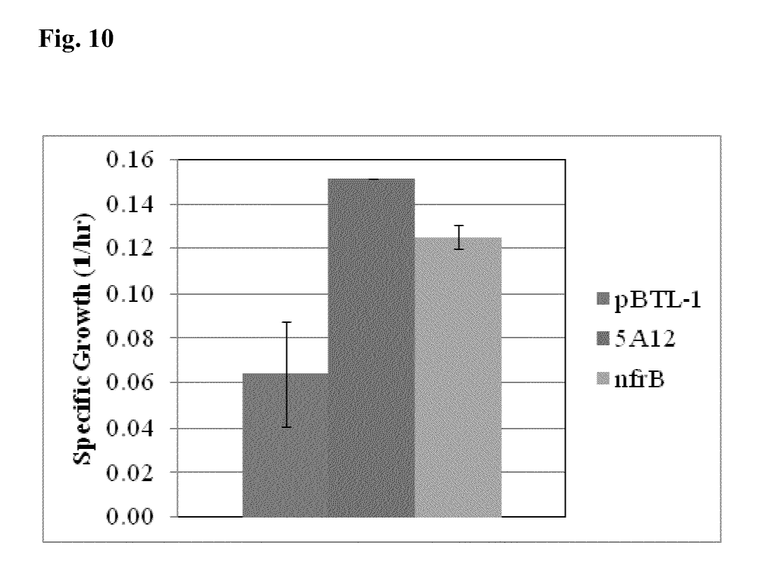

In one example, identification of gene nfrB as dominant contributing component towards tolerance from an exemplary clone, Clone 5A12. Cultures were grown in 5 ml MOPS minimal media, 100 μg carbenicillin/ml, and 1 g furfural/1 supplemented in 15 ml conical tubes at 37° C. The optical density of 1 ml culture was measured with a spectrophotometer at 600 nm. A natural log transformation of the data was then calculated and then a regression line was fit to the data to calculate specific growth, which is proportional to the culture doubling time. The blank vector, pBTL-1, served as the control. Clone 5A12 contains the gene nfrB as well as approximately 2200 by of surrounding genomic sequencing. The nfrB subclone was produced by standard cloning techniques with primers designed to encode for the gene. The resulting cloned DNA was then ligated to pBTL-1 vector. FIG. 10 represents a histogram plot of data obtained from this clone. This data supports that the expression of nfrB contributes to aldehyde tolerance. Compared to control, Clone 5A12 has a higher specific growth. The nfrB subclone also contributes to increasing aldehyde tolerance.

### Example 7

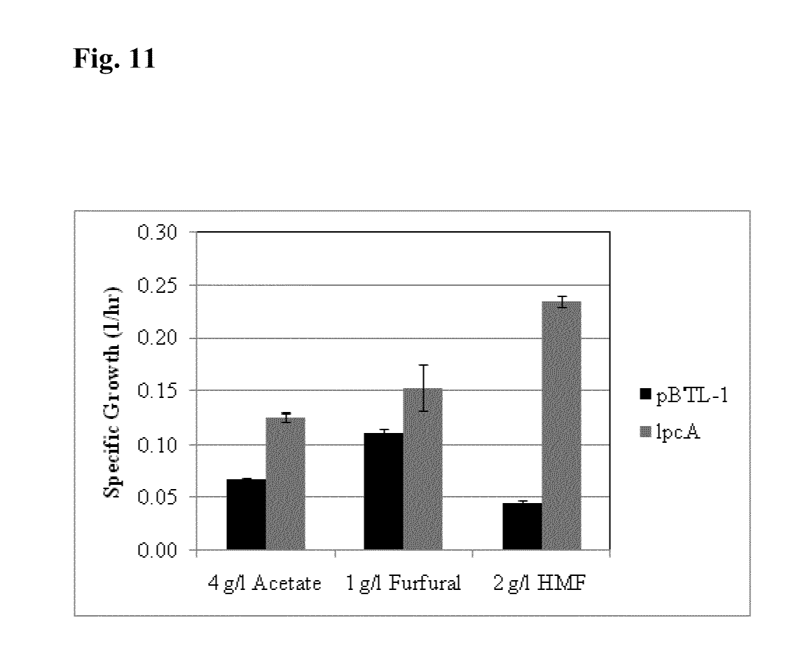

In other methods, a summary of the data in the tables obtained from studies performed herein is represented in FIG. 11. Here, confirmation of lpcA conferring tolerance to furfural and HMF (aldehydes) and acetate (acid) were observed. Cultures were grown in 10 ml MOPS minimal media, 100 μg carbenicillin/ml, and either 1 g furfural/1 supplemented, 2 g HMF/1, or 4 g acetate/1, in 15 ml conical tubes at 37° C. The optical density of 1 ml culture was measured with a spectrophotometer at 600 nm. A natural log transformation of the data was then calculated and then a regression line was fit to the data to calculate specific growth, which is proportional to the culture doubling time. The blank vector, pBTL-1, served as the control. The lpcA gene was cloned into the pBTL-1 using standard cloning techniques and primers designed to amplify the gene from template DNA.

This data supports that lpcA confers tolerance to a variety of low molecular weight organic compounds, both aldehydes and acids. Under all conditions tested, specific growth is improved when lpcA is overexpressed using the pBTL-1 vector system.

### Example 8

In another exemplary method, (data not shown) a circle plot after 72 hours in 1.75 g/L selection was compiled. Peaks represent clones found after SCALEs selection and analysis. Peak size represents relative fitness. Peak location represents location on E. coli genome. Peak color represents size of clone: blue 8 kb, green 4 kb, yellow 2 kb, and red 1 kb. In addition, growth rate of high-fitness clones compared to control were analyzed using various gene inductions (data not shown).

### Example 9

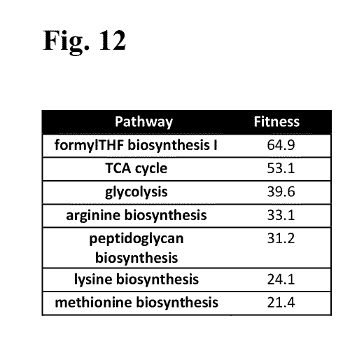

Top pathway fitness. Individual gene fitness were determined by analyzing the clones found in the SCALEs data (see FIG. 12). Multiple clones may contain the same gene or part of a gene. To calculate the gene fitness per clone, the clone fitness was multiplied by the fraction of the gene contained in the clone; this was then divided by the length of the gene. Once this was done for all clones that contained the gene, these were summed to yield the total gene fitness. This process was repeated for every gene in the ecocyc.org database for E. coli K12 MG1655. Subsequently, pathway fitness was determined by summing the individual gene fitness for all the genes in a particular pathway. Here, from the 1.75 g/L selection, formylTHF biosynthesis genes were found to be important to acetate tolerance. Arginine and methionine are also important pathway targets based on these findings.

### Example 10

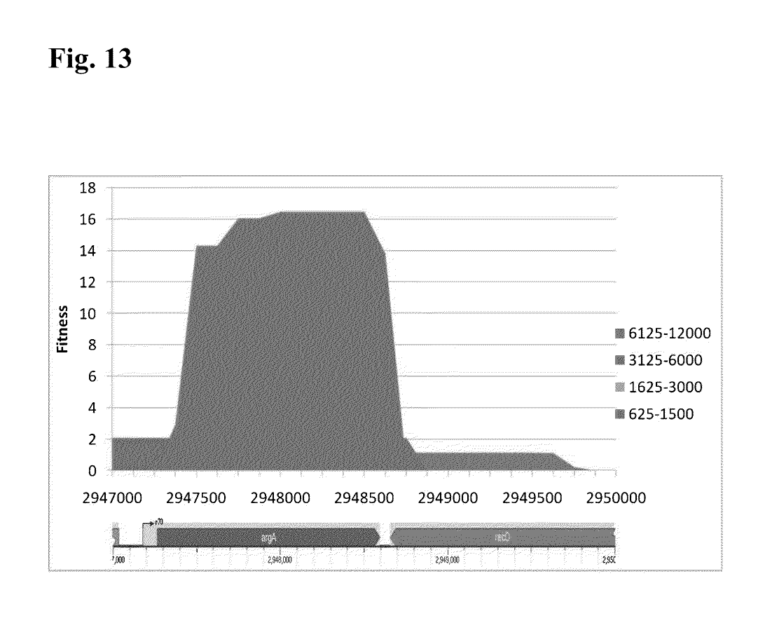

Blow-up regions of the E. coli genome were evaluated and plotted (see FIG. 13). Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrate, the region of the genome that contains the gene argA from the 1.75 g/L SCALEs selection and analysis.

### Example 11

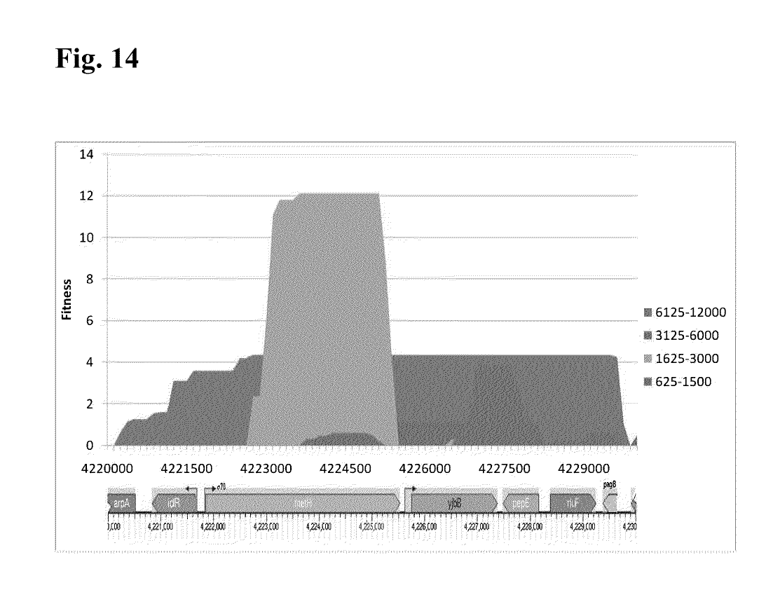

Blow-up regions of the E. coli genome were evaluated and plotted (see FIG. 14). Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrate, the region of the genome that contains the gene metH from the 1.75 g/L SCALEs selection and analysis.

### Example 12

FIG. 15 represents results from pilot selection. Randomly selected clones from the furfural aldehyde selection demonstrate a high frequency for the lgt-thyA operon.

TABLE 1
1.75 g/L Acetate Selection

| | | | | | |
|---|---|---|---|---|---|
| 100625 | 101875 | 1250 | 22.84 | murC†, murG | UDP-N-acetylmuramate-alanine ligase |
| 4353125 | 4354625 | 1500 | 15.19 | yjdL†, cadA | dipeptide transporter |
| 4352875 | 4354625 | 1750 | 12.33 | yjdL*, cadA | dipeptide transporter |
| 2947500 | 2948750 | 1250 | 11.39 | argA†, recD | N-acetylglutamate synthase |
| 4344000 | 4345750 | 1750 | 8.29 | fumB†, dcuB | fumarase B |

2.5 g/L Acetate Selection

| | | | | | |
|---|---|---|---|---|---|
| 4353125 | 4354625 | 1500 | 32.27 | yjdL†, cadA | dipeptide transporter |
| 4343625 | 4345625 | 2000 | 31.59 | fumB†, dcuB | fumarase B |
| 4352875 | 4354625 | 1750 | 18.94 | yjdL*, cadA | dipeptide transporter |
| 4344000 | 4345750 | 1750 | 12.88 | fumB†, dcuB | fumarase B |
| 4352625 | 4356625 | 4000 | 11.38 | yjdL*, cadA† | dipeptide transporter, |
| 100625 | 101875 | 1250 | 11.13 | murC† | UDP-N-acetylmuramate-alanine ligase |

*the entire gene is present in the clone.
†means that >80% of the gene is present in the clone

TABLE 2

| | | | |
|---|---|---|---|
| Gene Name | Fitness | Gene Product | Pathway |

1.75 g/L Selection

| | | | |
|---|---|---|---|
| Yjdl | 50.3 | dipeptide transporter | |
| murC | 23.7 | UDP-N-acetylmuramate-alanine ligase | peptidoglycan biosynthesis III |
| fumB | 19.6 | fumerase B | TCA cycle |
| cadA | 15.1 | lysine decarboxylase | lysine degradation |
| argA | 14.9 | N-acetylglutamate synthase | arginine biosynthesis |
| meth | 13.7 | homocysteine transmethylase | formylTHF biosynthesis 1 |

2.5 g/L Selection

| | | | |
|---|---|---|---|
| Yjdl | 78.0 | dipeptide transporter | |
| murC | 11.9 | UDP-N-acetylmuramate-alanine ligase | peptidoglycan biosynthesis III |
| fumB | 21.2 | fumerase B | TCA cycle |
| cadA | 24.7 | lysine decarboxylase | lysine degradation |
| lpcA | 9.7 | D-sedoheptulose 7-phosphate isomerase | ADP-L-glycero-b-D-manno-heptose |
| | | | biosynthesis |

TABLE 3
Acid Tolerance (Acetate Selection): Data from aerobic growth in 4 g/L acetate

| | SCALEs | Specific | Standard | | Final OD₆₀₀ | |
|---|---|---|---|---|---|---|
| Gene | Fitness | Growth (hr⁻¹) | Deviation | n | (24 hr) | Comment |
| pBTL-1 | N/A | 0.067 | 0.001 | 3 | 0.111 | |
| Control | | | | | | |
| pBTL-1 | Get from | 0.126 | 0.004 | 3 | 0.324 | SCALEs selection |
| lpcA | Nich | | | | | performed in pBTL- |
| | | | | | | 1 vector |

TABLE 4
Aldehyde Tolerance (Furfural Selection): Data from aerobic growth in 1 g/L furfural

| | | | | | Final | |
|---|---|---|---|---|---|---|
| | SCALEs | Specific | Standard | | OD₆₀₀ | |
| Gene | Fitness | Growth (hr⁻¹) | Deviation | n | (24 hr) | Comment |
| pBTL-1 | N/A | 0.111 | 0.003 | 3 | 0.586 | |
| Control | | | | | | |
| pBTL-1 | 11.9 | 0.153 | 0.022 | 3 | 0.918 | SCALEs selection |
| lpcA | | | | | | performed in |
| | | | | | | pSMART LCK |
| | | | | | | vector, confirmation studies |
| | | | | | | performed in |
| | | | | | | pBTL-1 |

TABLE 5
Aldehyde Tolerance (Furfural Selection): Data from
aerobic growth in 2 g/L hydroxymethyl furfural

| | | Specific | | | Final | |
|---|---|---|---|---|---|---|
| | SCALEs | Growth | Standard | | OD₆₀₀ | |
| Gene | Fitness | (hr⁻¹) | Deviation | n | (24 hr) | Comment |
| pBTL-1 | N/A | 0.045 | 0.002 | 3 | 0.170 | |
| Control | | | | | | |
| pBTL-1 | See fitness | 0.234 | 0.005 | 3 | 1.014 | SCALEs selection performed |
| lpcA | for furfural | | | | | in pSMART LCK vector, |
| | selection | | | | | confirmation studies |
| | | | | | | performed in pBTL-1 |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A composition for increasing tolerance for biomass hydrolysate by a microorganism comprising a vector capable of inducing one or more genes of one or more pathways capable of modulating tolerance of biomass hydrolysate by the microorganism, induction of the one or more genes of one or more pathways modulates the tolerance to low molecular weight organic compounds found in biomass hydrolysate by the microorganism.

2. The composition of claim 1, wherein low molecular weight organic compounds comprise short chain organic acids of ten carbons or less, cyclic organic acids of ten carbons or less, organic compounds having a terminal carbonyl group or formyl group side chain and any combination thereof.

3. The composition of claim 1, wherein the vector modulates one or more genes of one or more pathways to increase low molecular weight organic compound tolerance by the microorganism compared to a control microorganism not having the vector.

4. The composition of claim 3, wherein short chain organic acids comprise formic acid, acetic acid, citric acid, propionic acid, pyruvic acid, oxalic acid, succinic acid, malonic acid, levulinic acid or a combination thereof.

5. The composition of claim 1, wherein the low molecular weight organic compounds are selected from the group consisting of formaldehyde, acetaldehyde, and butyraldehyde, furan-2-carbaldehyde (commonly known as furfural), 5-(hydroxymethyl)-2-furaldehyde (commonly known as hydroxymethylfurfural) and phenolic aldehydes like benzaldehyde, 4-hydroxy-3-methoxybenzaldehyde (commonly known as vanillin), and (2E)-3-phenylprop-2-enal (commonly known as cinnamaldehyde) or a combination thereof.

6. The composition of claim 1, wherein modulating the tolerance comprises increasing growth rate of the microorganism by 5% or more; or 15% or more; or 25% or more; or 35% or more; or over 100% or more relative to a control microorganism.

7. The composition of claim 1, wherein one or more genes comprise one or more of lpcA, lgt, nfrB, thyA or a combination thereof.

8. The composition of claim 1, wherein one or more genes comprise one or more of lpcA, murC, fumB, cadA, yjdL, argA, metH or a combination thereof.

9. The composition of claim 1, wherein the vector comprises pEZseq (Lucigen) cloning systems, pSMART cloning systems (Lucigen), pACYCDuet cloning systems (Novagen) pBMT-1, pBMT-2, pBMT-3, pBMT-4, pBMT-5, pBMT-6, pBT-1, pBT-2, pBT-3, pBT-4, pBT-5, pBT-6, pBMTB-1, pBMTB-2, pBMTB-3, pBMTB-4, pBMTB-5, pBMTB-6, pBTB-1, pBTB-2, pBTB-3, pBTB-4, pBTB-5, pBTB-6, pBMTL-1, pBMTL-2, pBMTL-3, pBMTL-4, pBMTL-5 pBMTL-6, pBTL-1, pBTL-2, pBTL-3, pBTL-4, pBTL-5, pBTL-6 or any vector capable of inducing the one or more genes.

10. The composition of claim 1, wherein the vector comprises a vector capable of stably integrating into the genome of the microorganism and the genes are cloned into the microorganism, the genes selected from the group consisting of lpcA, lgt, nfrB, thyA, murC, fumB, cadA, yjdL, argA, metH and a combination thereof.

11. The composition of claim 1, further comprising supplementary amino acids.

12. A method for modulating tolerance of biomass hydrolysates by a microorganism comprising modulating one or more genes in one or more pathways that modulate tolerance to low molecular weight organic compounds found in biomass hydrolysates by the microorganism.

13. The method of claim 12, wherein modulating the tolerance comprises increasing growth rate of the microorganism by 5% or more; or 15% or more; or 25% or more; or 35% or more; or over 100% or more relative to a control microorganism.

14. The method of claim 12, wherein one or more pathways comprises formylTHF biosynthesis I, glycolysis, arginine biosynthesis, peptidoglycan biosynthesis, lysine biosynthesis methionine biosynthesis or combinations thereof.

15. The method of claim 12, wherein the one or more pathways comprise formylTHF biosynthesis I.

16. The method of claim 12, wherein modulating one or more genes comprises modulating one or more gene comprising lpcA, lgt, nfrB, thyA, murC, fumB, cadA, yjdL, argA, metH and a combination thereof.

17. A composition for increasing tolerance for biomass hydrolysate by a microorganism comprising a vector capable of inducing one or more genes selected from the group consisting of lpcA, lgt, nfrB, thyA, murC, fumB, cadA, yjdL, argA, metH and a combination thereof for modulating tolerance of biomass hydrolysate by the microorganism, induction of the one or more genes modulates the tolerance to low molecular weight organic compounds found in biomass hydrolysate by the microorganism.

18. A method for modulating tolerance for biomass conversion by a microorganism comprising:
a) obtaining a vector capable of inducing one or more genes selected from the group consisting of lpcA, lgt, nfrB, thyA, murC, fumB, cadA, yjdL, argA, metH and a combination thereof wherein induction of the one or more genes modulates tolerance of low molecular weight organic compounds by the microorganism; and
b) introducing the vector to a culture of the microorganism.

19. The method of claim 18, wherein the low molecular weight organic compounds are selected from the group consisting of formic acid, acetic acid, citric acid, propionic acid, pyruvic acid, oxalic acid, succinic acid, malonic acid, levulinic acid, formaldehyde, acetaldehyde, and butyraldehyde, furan-2-carbaldehyde (commonly known as furfural), 5-(hydroxymethyl)-2-furaldehyde (commonly known as hydroxymethylfurfural) and phenolic aldehydes like benzaldehyde, 4-hydroxy-3-methoxybenzaldehyde (commonly known as vanillin), and (2E)-3-phenylprop-2-enal (commonly known as cinnamaldehyde) and a combination thereof.

20. A kit comprising, one or more vectors capable of modulating one or more genes selected from the group consisting of lpcA, lgt, nfrB, thyA, murC, fumB, cadA, yjdL, argA, metH and a combination thereof; and at least one container and a culture of microorganisms.

21. The kit of claim 20, wherein the culture of microorganisms is a bacterial culture.

22. The kit of claim 20, wherein the culture comprises Escherichia. coli.
